SEQUENCE LISTING (43 sequences)

SEQ ID NO: 1 - DNA, 21 bp, Artificial Sequence
  Description of Artificial Sequence Synthetic primer
cttgtccatcactggcatctg

SEQ ID NO: 2 - DNA, 20 bp, Artificial Sequence
  Description of Artificial Sequence Synthetic primer
cggtgaataggcagacagac

SEQ ID NO: 3 - DNA, 21 bp, Artificial Sequence
  Description of Artificial Sequence Synthetic primer
gccaagattgatagcttgtgc

SEQ ID NO: 4 - DNA, 21 bp, Artificial Sequence
  Description of Artificial Sequence Synthetic primer
gtcagatttgttgctccaggc

SEQ ID NO: 5 - DNA, 22 bp, Artificial Sequence
  Description of Artificial Sequence Synthetic primer
gcagtattattaagtagccctg

SEQ ID NO: 6 - DNA, 17 bp, Artificial Sequence
  Description of Artificial Sequence Synthetic primer
cgaaggcaccaaagctg

SEQ ID NO: 7 - DNA, 21 bp, Artificial Sequence
  Description of Artificial Sequence Synthetic primer
cagtttgctttgctgggcctt

SEQ ID NO: 8 - DNA, 18 bp, Artificial Sequence
  Description of Artificial Sequence Synthetic primer
ggcaatggataaggccga

SEQ ID NO: 9 - DNA, 22 bp, Artificial Sequence
  Description of Artificial Sequence Synthetic oligonucleotide
cccacagatatccagaaccctg

SEQ ID NO: 10 - DNA, 23 bp, Artificial Sequence
  Description of Artificial Sequence Synthetic oligonucleotide
  modified_base (21)..(21): a, c, t, g, unknown or other
gaggagcggatgcataggctngg

SEQ ID NO: 11 - DNA, 23 bp, Artificial Sequence
  Description of Artificial Sequence Synthetic oligonucleotide
  modified_base (21)..(21): a, c, t, g, unknown or other
ggattgaggagcggatgcatngg

SEQ ID NO: 12 - DNA, 26 bp, Artificial Sequence
  Description of Artificial Sequence Synthetic primer
agtgtaataccttgcagcaccagagc

SEQ ID NO: 13 - DNA, 25 bp, Artificial Sequence
  Description of Artificial Sequence Synthetic primer
ttgctccaggccacagcactgttgc

SEQ ID NO: 14 - DNA, 400 bp, Unknown
  Description of Unknown Left Homology Arm sequence
actaagactatctgctcagcacttctggtgacccaaaagggtctgaggacaggagctcagagttgggtcagctgtccaggtactcagggttgtcacaggcaaaactgctggaactcagggcagcattgcaaatgccacgccgctctcagggccccttgcctgccgctggaattaaacccacccagatcttggaaactctgccctggacccttctcaataagtccatgagaaatcaaactctttcctttatgcgacactggattttccacaaagtaaaatcaagatgagtaaagatgtggtttctagatagtgcctgaaaaagcagagaccatggtgtcaggcgtcaccacttgggcctataaaagctgccacaagacgccaaggccacaagccacacagc

SEQ ID NO: 15 - DNA, 400 bp, Unknown
  Description of Unknown Right Homology Arm sequence
ctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggtgagtgtcggctagccagggtcctagctatgagggctccagggtgggtgattcccaagatgaggtcatgagcaggctgggcctggtcctaagatgcctgtaggtcaggaaaaatctccatggaccaaggcccggcccagccatgagggagagaggagctgggctggggggctcagcactgtggatggacctatggaggtgtctggcagactccccagggacta

SEQ ID NO: 16 - RNA, 20 nt, Artificial Sequence
  Description of Artificial Sequence Synthetic oligonucleotide
gaggagcggaugcauaggcu

SEQ ID NO: 17 - RNA, 20 nt, Artificial Sequence
  Description of Artificial Sequence Synthetic oligonucleotide
ggauugaggagcggaugcau

SEQ ID NO: 18 - protein, 18 aa, Thosea asigna virus
EGRGSLLTCGDVEENPGP

SEQ ID NO: 19 - protein, 21 aa, Artificial Sequence
  Description of Artificial Sequence Synthetic peptide
GSGEGRGSLLTCGDVEENPGP

SEQ ID NO: 20 - protein, 63 aa, Artificial Sequence
  Description of Artificial Sequence Synthetic polypeptide
GSGSRVTELLYRMKRAETYCPRPLLAIHPTEARHKQKIVAPVKQLLNFDLLKLAGDVESNPGP

SEQ ID NO: 21 - protein, 19 aa, Amphimedon sp.
LLCFLLLLLSGDVELNPGP

SEQ ID NO: 22 - protein, 20 aa, Amphimedon sp.
HHFMFLLLLLAGDIELNPGP

SEQ ID NO: 23 - protein, 20 aa, Saccoglossus kowalevskii
WFLVLLSFILSGDIEVNPGP

SEQ ID NO: 24 - protein, 20 aa, Branchiostoma floridae
KNCAMYMLLLSGDVETNPGP

SEQ ID NO: 25 - protein, 20 aa, Branchiostoma floridae
MVISQLMLKLAGDVEENPGP

SEQ ID NO: 26 - protein, 22 aa, Artificial Sequence
  Description of Artificial Sequence Synthetic peptide
GSGATNFSLLKQAGDVEENPGP

SEQ ID NO: 27 - protein, 23 aa, Artificial Sequence
  Description of Artificial Sequence Synthetic peptide
GSGQCTNYALLKLAGDVESNPGP

SEQ ID NO: 28 - protein, 8 aa, Artificial Sequence
  Description of Artificial Sequence Synthetic peptide
  MOD_RES (2)..(2): Any amino acid
  MOD_RES (4)..(4): Any amino acid
DXEXNPGP

SEQ ID NO: 29 - DNA, 90 bp, Homo sapiens
  CDS (37)..(90)
atcatgtcctaaccctgatcctcttgtcccacagatatccagaaccctgaccctgccgtgtaccagctgagagactctaaatccagtgac

SEQ ID NO: 30 - protein, 18 aa, Homo sapiens
IQNPDPAVYQLRDSKSSD

SEQ ID NO: 31 - DNA, 60 bp, Artificial Sequence
  Description of Artificial Sequence Synthetic oligonucleotide
atgtcctaaccctgatcctcttgtcccacagatatccagaagcctgaccctgccgtgtac

SEQ ID NO: 32 - DNA, 49 bp, Artificial Sequence
  Description of Artificial Sequence Synthetic oligonucleotide
atgtcctaaccctgatcctcttgtccagaagcctgaccctgccgtgtac

SEQ ID NO: 33 - DNA, 50 bp, Artificial Sequence
  Description of Artificial Sequence Synthetic oligonucleotide
atgtcctaaccctgatcctcttgtcccagaagcctgaccctgccgtgtac

SEQ ID NO: 34 - DNA, 60 bp, Artificial Sequence; identical to SEQ ID NO: 31
  Description of Artificial Sequence Synthetic oligonucleotide

SEQ ID NO: 35 - DNA, 28 bp, Artificial Sequence
  Description of Artificial Sequence Synthetic oligonucleotide
atgtcctaaccctgatcctgccgtgtac

SEQ ID NO: 36 - DNA, 60 bp, Artificial Sequence
  Description of Artificial Sequence Synthetic oligonucleotide
atgtcctaaccctgatcctcttgtcccacaagatatccagaagcctgaccctgccgtgta

SEQ ID NO: 37 - DNA, 58 bp, Artificial Sequence
  Description of Artificial Sequence Synthetic oligonucleotide
atgtcctaaccctgatcctcttgtcccacatatccagaagcctgaccctgccgtgtac

SEQ ID NO: 38 - DNA, 41 bp, Artificial Sequence
  Description of Artificial Sequence Synthetic oligonucleotide
atgtcctaaccctgatccagaagcctgaccctgccgtgtac

SEQ ID NO: 39 - DNA, 59 bp, Artificial Sequence
  Description of Artificial Sequence Synthetic oligonucleotide
atgtcctaaccctgatcctcttgtcccacgatatccagaagcctgaccctgccgtgtac

SEQ ID NO: 40 - DNA, 52 bp, Artificial Sequence
  Description of Artificial Sequence Synthetic oligonucleotide
atgtcctaaccctgatcctcttgtcccacagaagcctgaccctgccgtgtac

SEQ ID NO: 41 - DNA, 28 bp, Artificial Sequence
  Description of Artificial Sequence Synthetic oligonucleotide
atgtcctaaccctgaccctgccgtgtac

SEQ ID NO: 42 - DNA, 53 bp, Artificial Sequence
  Description of Artificial Sequence Synthetic oligonucleotide
atgtcctaaccctgatcctcttgtcccacaagaagcctgaccctgccgtgtac

SEQ ID NO: 43 - DNA, 22 bp, Artificial Sequence
  Description of Artificial Sequence Synthetic oligonucleotide
atgtcctaaccctgccgtgtac

(19) United States
(12) Patent Application Publication
(10) Pub. No.: US 2023/0340067 A1
(43) Pub. Date: Oct. 26, 2023

(54) METHODS OF GENERATING AN ACTIVATION INDUCIBLE EXPRESSION SYSTEM IN IMMUNE CELLS

(71) Applicant: St. Jude Children's Research Hospital, Inc., Memphis, TN (US)

(72) Inventors: Stephen GOTTSCHALK, Germantown, TN (US); Giedre KRENCIUTE, Memphis, TN (US); Zelda ODE, Amsterdam (NL)

(73) Assignee: St. Jude Children's Research Hospital, Inc., Memphis, TN (US)

(21) Appl. No.: 18/003,093

(22) PCT Filed: Jun. 25, 2021
(86) PCT No.: PCT/US21/39029

§ 371 Date: Dec. 22, 2022

Related U.S. Application Data
(60) Provisional application No. 63/044,797, filed on Jun. 26, 2020.

(51) Int. Cl. C07K 14/705; C07K 14/725; C07K 14/73; C12N 5/0783; C12N 15/11; C12N 9/22; C12N 15/90

(52) U.S. Cl. CPC C07K 14/70521; C07K 14/7051; C07K 14/70514; C07K 14/70517; C12N 5/0636; C12N 15/11; C12N 9/22; C12N 15/907; C07K 2319/03; C12N 2310/20; C12N 2800/80

(57) ABSTRACT

The present invention provides methods of genetically modifying an immune cell such that the immune cell expresses a transgene in an activation dependent manner. The application also provides genetically modified immune cells prepared using such methods, and the uses of the genetically modified immune cells in immunotherapy (e.g., adoptive cell therapy) for treatment of a disease such as cancer, autoimmune disease or infectious disease.

### CROSS-REFERENCE TO RELATED APPLICATIONS

This application claims priority to U.S. Provisional Patent Application No. 63/044,797, filed Jun. 26, 2020, the disclosure of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 24, 2021, is named 243734_000148_SL.txt and is 12,623 bytes in size.

### FIELD OF THE INVENTION

The application relates to methods of genetically modifying an immune cell such that the immune cell expresses a transgene in an activation dependent manner. The application also relates to genetically modified immune cells prepared using such methods, and the uses of the genetically modified immune cells in immunotherapy (e.g., adoptive cell therapy) for treatment of a disease such as cancer, autoimmune disease or infectious disease.

### BACKGROUND

Adoptive cell therapies are widely being explored for the treatment of several clinical indications including multiple types of cancer and autoimmune diseases [1, 2]. One therapy involves adoptive transfer of patient-derived T cells that have been engineered to express chimeric antigen receptors (CARs) specific for tumor-associated antigens. CAR engineered T cells are then infused back into the patient and are able to recognize and kill tumor cells expressing targeted antigens on their surface. CAR T cell therapy has produced outstanding results in CD19-positive hematological B-cell malignancies resulting in complete response rates of about 70% in relapsed/refractory pediatric B-cell acute lymphoblastic leukemia (ALL) patients [3-5].

Current preclinical and clinical production of CAR T cells relies to a great extent on T cell transduction using viral vectors (e.g. retrovirus, lentivirus) to deliver transgenes of interest. However, a CAR transgene delivered to a T cell by viral transduction is subjected to random integration into the host DNA. This can lead to unpredictable and variable expression of the transgene. In addition, random integration could lead to a malignant transformation if the transgene is integrated into an oncogenic locus [6, 7]. Finally, viral vector production is time-consuming (>6 months), expensive and poses biosafety challenges [8-12].

Genetic engineering approaches that aim to integrate a therapeutic gene into a targeted locus have the potential to solve the problems associated with random integration. Site-specific gene integration can be achieved with the use of gene-editing tools (e.g., Clustered regularly interspaced short palindromic repeats (CRISPR)-Cas9, Transcription activator-like effector nuclease (TALEN), Zinc finger nuclease (ZFN), Meganucleases), which results in DNA double-strand breaks (DSBs) and homology-directed repair (HDR) when a donor DNA template is provided. This approach is known as gene knock-in (KI). More and more KI studies are emerging using CRISPR-Cas9 in human T cells to develop clinically useful engineered T cells. However, CRISPR-Cas9-mediated knock-in methods remain to be optimized for better knock-in efficiencies and reduced toxicity. Various delivery approaches have been employed to deliver the donor DNA template [13-17], including the use of viral vectors. Viral-vector based approaches for the delivery of donor DNA template can be time consuming, expensive and labor-intensive because it requires cloning template DNA into the appropriate vector and producing a high titer viral supernatant prior to gene editing. Thus, there exists a need for an improved knock-in method that is efficient, fast, and inexpensive.

Further, it is desirable that the function of adoptively transferred T cells can be enhanced in an activation dependent manner. While several systems have been developed to couple gene expression to T-cell activation, most of them use virus-encoded regulatory elements, which are complex [28]. Thus, there exists a need for an improved approach that allows transgene expression to be tightly controlled by T-cell activation.

This present invention addresses these and other related needs.

### SUMMARY OF THE INVENTION

The present invention provides, in various aspects, methods of genetically modifying an immune cell (e.g., T cell, natural killer (NK) cell) such that the immune cell expresses a transgene in an activation dependent manner. Further provided are genetically modified immune cells, pharmaceutical compositions, and methods of using such cells or compositions in treating a disease (e.g., cancer, autoimmune disease, or infectious disease) in a subject.

In some aspects, provided herein is a method of genetically modifying an immune cell, comprising introducing into the immune cell at least one transgene, wherein the at least one transgene is inserted at a target site of interest within the immune cell genome. The target site of interest preferably has an promotor which is inducible by activation of the immune cell. In some embodiments, the at least one transgene is inserted such that expression of the at least one transgene is under the control of the promoter at the target site of interest. In some embodiments, expression of the endogenous gene at the target site of interest is reduced or abolished.

In one aspect, provided herein is a method of genetically modifying an immune cell, comprising introducing into the immune cell at least one transgene, wherein the at least one transgene is inserted at the interleukin 13 (IL-13) gene locus within the immune cell genome. In some embodiments, the at least one transgene is inserted such that expression of the at least one transgene is under the control of the endogenous IL-13 promoter. In some embodiments, expression of the endogenous IL-13 is reduced or abolished.

In some embodiments, the at least one transgene encodes a therapeutic molecule. In some embodiments, the therapeutic molecule is selected from a chimeric antigen receptor (CAR), a cytokine, a cytokine receptor, a chimeric cytokine receptor, a switch receptor, a chemokine, an antibody, and a bispecific antibody. In some embodiments, the therapeutic molecule is a chimeric antigen receptor (CAR). In some embodiments, the therapeutic molecule is an IL13Rα2-specific CAR. In some embodiments, the therapeutic molecule is a cytokine. In some embodiments, the therapeutic molecule is interleukin 15 (IL-15). In some embodiments, the therapeutic molecule is a bispecific T cell engager (BiTE). In some embodiments, the therapeutic molecule is a BiTE that binds specifically to Tumor Endothelial Marker 8 (TEM8) and CD3.

In some embodiments, the 5′ end of the at least one transgene comprises a sequence encoding a self-cleaving peptide and/or an internal ribosomal entry site (IRES). In some embodiments, the self-cleaving peptide is a 2A peptide. In some embodiments, the 3′ end of the at least one transgene comprises a polyadenylation (polyA) sequence. In some embodiments, the at least one transgene is operatively linked to at least one insulator and/or enhancer sequence.

In some embodiments, the insertion of the at least one transgene is mediated by a site-specific nuclease. In some embodiments, the site-specific nuclease comprises a Cas protein and a guide RNA capable of targeting the IL-13 gene locus. In some embodiments, the Cas protein is a Cas9 protein.

In some embodiments, the guide RNA is a single guide RNA (sgRNA). In some embodiments, the guide RNA comprises the nucleotide sequence of GAGGAGCGGAUGCAUAGGCU (SEQ ID NO: 16) or GGAUUGAGGAGCGGAUGCAU (SEQ ID NO: 17), or a nucleotide sequence having at least 80% identity thereof.

In some embodiments, the at least one transgene is introduced into the immune cell via a donor polynucleotide. In some embodiments, the donor polynucleotide is a non-viral polynucleotide. In some embodiments, the donor polynucleotide is a single-stranded DNA, a double-stranded DNA, or a plasmid. In some embodiments, the donor polynucleotide is a double-stranded DNA. In some embodiments, the donor polynucleotide comprises a 5′ homology arm and a 3′ homology arm. In some embodiments, the 5′ homology arm and the 3′ homology arm comprise sequences flanking the insertion locus of the at least one transgene. In some embodiments, the 5′ homology arm and the 3′ homology arm each have between about 100 to about 1500 base pairs (bp) in length. In some embodiments, the 5′ homology arm and the 3′ homology arm each have about 400 bp, about 800 bp or about 1200 bp in length. In some embodiments, the 5′ homology arm in the donor polynucleotide comprises the nucleotide sequence of SEQ ID NO: 14, or a nucleotide sequence having at least 80% identity thereof, or a fragment thereof. In some embodiments, the 3′ homology arm in the donor polynucleotide comprises the nucleotide sequence of SEQ ID NO: 15, or a nucleotide sequence having at least 80% identity thereof, or a fragment thereof.

In some embodiments, the site-specific nuclease and/or the donor polynucleotide is introduced to the immune cell via a physical means. In some embodiments, the physical means is electroporation, microinjection, magnetofection, ultrasound, a ballistic or hydrodynamic method, or a combination thereof. In some embodiments, the physical means is electroporation. In some embodiments, the electroporation is conducted by mixing about 0.25×10⁶-2.0×10⁶ immune cells with about 1 μg-3 μg of donor polynucleotide. In some embodiments, the electroporation is conducted by mixing about 1.0×10⁶ immune cells with about 2 μg of donor polynucleotide.

In some embodiments, the immune cell is allowed to recover for about 4-12 days after introduction of the at least one transgene.

In some embodiments, the method further comprises activating the immune cell. In some embodiments, expression of the at least one transgene is increased after the immune cell is activated.

In various embodiments, the immune cell is a T cell. In some embodiments, the T cell is an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (TSCM), a naïve T-cell, an effector T-cell, a T-helper cell, or a regulatory T-cell (Treg). In some embodiments, the T cell is activated by CD3, CD28 and/or CD2 stimulation.

In various embodiments, the immune cell is a natural killer (NK) cell. In some embodiments, the NK cell is activated by inhibition of a inhibitory receptor on the NK cell, feeder cells, interferons and/or macrophage-derived cytokines.

In various embodiments, the immune cell is an allogeneic cell. In various embodiments,

the immune cell is an autologous cell. In various embodiments, the immune cell is derived from a blood, marrow, tissue, or a tumor sample.

In another aspect, provided herein is a genetically modified immune cell prepared according to the method described above.

In another aspect, provided herein is a genetically modified immune cell, comprising at least one transgene inserted at the interleukin 13 (IL-13) gene locus within the immune cell genome. In some embodiments, expression of the at least one transgene is under the control of the endogenous IL-13 promoter. In some embodiments, expression of the endogenous IL-13 is reduced or abolished.

In some embodiments of the genetically modified immune cell, the at least one transgene encodes a therapeutic molecule. In some embodiments, the therapeutic molecule is selected from a chimeric antigen receptor (CAR), a cytokine, a cytokine receptor, a chimeric cytokine receptor, a switch receptor, a chemokine, an antibody, and a bispecific antibody. In some embodiments, the therapeutic molecule is a chimeric antigen receptor (CAR). In some embodiments, the therapeutic molecule is an IL13Rα2-specific CAR. In some embodiments, the therapeutic molecule is a cytokine. In some embodiments, the therapeutic molecule is interleukin 15 (IL-15). In some embodiments, the therapeutic molecule is a bispecific T cell engager (BiTE). In some embodiments, the therapeutic molecule is a BiTE that binds specifically to Tumor Endothelial Marker 8 (TEM8) and CD3.

In some embodiments of the genetically modified immune cell, the 5′ end of the at least one transgene comprises a sequence encoding a self-cleaving peptide and/or an internal ribosomal entry site (IRES). In some embodiments, the self-cleaving peptide is a 2A peptide. In some embodiments, the 3′ end of the at least one transgene comprises a polyadenylation (polyA) sequence. In some embodiments, the at least one transgene is operatively linked to at least one insulator and/or enhancer.

In various embodiments of the genetically modified immune cell, the immune cell is a T cell. In some embodiments, the T cell is an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (TSCM), a naïve T-cell, an effector T-cell, a T-helper cell, or a regulatory T-cell (Treg). In various embodiments, the immune cell is an NK cell.

In various embodiments, the immune cell is an allogeneic cell. In various embodiments, the immune cell is an autologous cell.

In various embodiments, the immune cell is derived from a blood, marrow, tissue, or a tumor sample.

In one aspect, provided herein is a method of genetically modifying an immune cell, comprising introducing into the immune cell at least one transgene, wherein the at least one transgene is inserted at the granulocyte-macrophage colony-stimulating factor (GM-CSF) gene locus within the immune cell genome. In some embodiments, the at least one transgene is inserted such that expression of the at least one transgene is under the control of the endogenous GM-CSF promoter. In some embodiments, expression of the endogenous GM-CSF is reduced or abolished.

In some embodiments, the at least one transgene encodes a therapeutic molecule. In some embodiments, the therapeutic molecule is selected from a chimeric antigen receptor (CAR), a cytokine, a cytokine receptor, a chimeric cytokine receptor, a switch receptor, a chemokine, an antibody, and a bispecific antibody. In some embodiments, the therapeutic molecule is a chimeric antigen receptor (CAR). In some embodiments, the therapeutic molecule is an IL13Rα2-specific CAR. In some embodiments, the therapeutic molecule is a cytokine. In some embodiments, the therapeutic molecule is interleukin 15 (IL-15). In some embodiments, the therapeutic molecule is a bispecific T cell engager (BiTE). In some embodiments, the therapeutic molecule is a BiTE that has specificity for Tumor Endothelial Marker 8 (TEM8) and CD3.

In some embodiments, the 5′ end of the at least one transgene comprises a sequence encoding a self-cleaving peptide and/or an internal ribosomal entry site (IRES). In some embodiments, the self-cleaving peptide is a 2A peptide. In some embodiments, the 3′ end of the at least one transgene comprises a polyadenylation (polyA) sequence. In some embodiments, the at least one transgene is operatively linked to at least one insulator and/or enhancer sequence.

In some embodiments, the insertion of the at least one transgene is mediated by a site-specific nuclease. In some embodiments, the site-specific nuclease comprises a Cas protein and a guide RNA capable of targeting the GM-CSF gene locus. In some embodiments, the Cas protein is a Cas9 protein. In some embodiments, the guide RNA is a single guide RNA (sgRNA).

In some embodiments, the at least one transgene is introduced into the immune cell via a donor polynucleotide. In some embodiments, the donor polynucleotide is a non-viral polynucleotide. In some embodiments, the donor polynucleotide is a single-stranded DNA, a double-stranded DNA, or a plasmid. In some embodiments, the donor polynucleotide is a double-stranded DNA. In some embodiments, the donor polynucleotide comprises a 5′ homology arm and a 3′ homology arm. In some embodiments, the 5′ homology arm and a 3′ homology arm comprise sequences flanking the insertion locus of the at least one transgene. In some embodiments, the 5′ homology arm and the 3′ homology arm each have between about 100 to about 1500 base pairs (bp) in length. In some embodiments, the 5′ homology arm and the 3′ homology arm each have about 400 bp, about 800 bp or about 1200 bp in length.

In some embodiments, the site-specific nuclease and/or the donor polynucleotide is introduced to the immune cell via a physical means. In some embodiments, the physical means is electroporation, microinjection, magnetofection, ultrasound, a ballistic or hydrodynamic method, or a combination thereof. In some embodiments, the physical means is electroporation. In some embodiments, the electroporation is conducted by mixing about 0.25×10⁶-2.0×10⁶ immune cells with about 1 μg-3 μg of the donor polynucleotide. In some embodiments, the electroporation is conducted by mixing about 1.0×10⁶ immune cells with about 2 μg of the donor polynucleotide.

In some embodiments, the immune cell is allowed to recover for about 4-12 days after introduction of the at least one transgene.

In some embodiments, the method further comprises activating the immune cell. In some embodiments, expression of the at least one transgene is increased after the immune cell is activated.

In various embodiments, the immune cell is a T cell. In some embodiments, the T cell is an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (TSCM), a naïve T-cell, an effector T-cell, a T-helper cell, or a regulatory T-cell (Treg). In some embodiments, the immune cell is activated by CD3, CD28 and/or CD2 stimulation.

In various embodiments, the immune cell is an NK cell. In some embodiments, the NK cell is activated by inhibition of a inhibitory receptor on the NK cell, feeder cells, interferons and/or macrophage-derived cytokines.

In various embodiments, the immune cell is an allogeneic cell. In some embodiments, the immune cell is an autologous cell.

In various embodiments, the immune cell is derived from a blood, marrow, tissue, or a tumor sample.

In another aspect, provided herein is a genetically modified immune cell prepared according to the method described above.

In another aspect, provided herein is a genetically modified immune cell, comprising at least one transgene inserted at the granulocyte-macrophage colony-stimulating factor (GM-CSF) gene locus within the immune cell genome. In some embodiments, expression of the at least one transgene is under the control of the endogenous GM-CSF promoter. In some embodiments, expression of the endogenous GM-CSF is reduced or abolished.

In some embodiments, the at least one transgene encodes a therapeutic molecule. In some embodiments, the therapeutic molecule is selected from a chimeric antigen receptor (CAR), a cytokine, a cytokine receptor, a chimeric cytokine receptor, a switch receptor, a chemokine, an antibody, and a bispecific antibody. In some embodiments, the therapeutic molecule is a chimeric antigen receptor (CAR). In some embodiments, the therapeutic molecule is an IL13Rα2-specific CAR. In some embodiments, the therapeutic molecule is a cytokine. In some embodiments, the therapeutic molecule is interleukin 15 (IL-15). In some embodiments, the therapeutic molecule is a bispecific T cell engager (BiTE). In some embodiments, the therapeutic molecule is a BiTE that has specificity for Tumor Endothelial Marker 8 (TEM8) and CD3.

In some embodiments, the 5′ end of the at least one transgene comprises a sequence encoding a self-cleaving peptide and/or an internal ribosomal entry site (IRES). In some embodiments, the self-cleaving peptide is a 2A peptide. In some embodiments, the 3′ end of the at least one transgene comprises a polyadenylation (polyA) sequence. In some embodiments, the at least one transgene is operatively linked to at least one insulator and/or enhancer.

In various embodiments of the genetically modified immune cell, the immune cell is a T cell. In some embodiments, the T cell is an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (TSCM), a naïve T-cell, an effector T-cell, a T-helper cell, or a regulatory T-cell (Treg). In some embodiments, the immune cell is an NK cell.

In various embodiments of the genetically modified immune cell, the immune cell is an allogeneic cell. In some embodiments, the immune cell is an autologous cell.

In various embodiments of the genetically modified immune cell, the immune cell is derived from a blood, marrow, tissue, or a tumor sample.

In another aspect, provided herein is a pharmaceutical composition comprising the genetically modified immune cell described herein, and a pharmaceutically acceptable carrier.

In a further aspect, provided herein is a method of treating a disease in a subject in need thereof, comprising administering to the subject an therapeutically effective amount of the genetically modified immune cell described herein, or the pharmaceutical composition described herein. In some embodiments, the disease is a cancer, an autoimmune disease, or an infectious disease.

In some embodiments of the treatment method, the method comprises:
a) isolating T cells and/or NK cells from the subject;
b) genetically modifying said T cells and/or NK cells ex vivo by introducing into the T cells and/or NK cells at least one transgene, wherein the at least one transgene is inserted at the IL-13 gene locus or GM-CSF gene locus within the genome of the T cells and/or NK cells;
c) optionally, expanding and/or activating said T cells and/or NK cells before, after or during step (b); and
d) introducing the genetically modified T cells and/or NK cells into the subject.

In some embodiments of the treatment method, the subject is human.

These and other aspects of the present invention will be apparent to those of ordinary skill in the art in the following description, claims and drawings.

### BRIEF DESCRIPTION OF THE DRAWINGS

FIG. 1 shows various steps that can be optimized in transgene knock-in using non-viral DNA delivery: (i) target site and guide RNAs, (ii) transgene design, (iii) donor DNA length, DNA type (ssDNA, dsDNA or plasmid) and delivery method, (iv) detection and efficiency of the knock-in and (v) viability and performance of genetically engineered T cell containing the gene of interest.

FIGS. 2A-2C show components of non-viral transgene cassette for expression under endogenous promoter. FIG. 2A is a schematic of non-viral cassette encoding a gene of interest surrounded by a 2A/IRES at the 5′ end for cleavage GOI from endogenous gene, and poly(A) region at the 3′ end for complete termination; _{L}HA: left homology arm, _{R}HA: right homology arm, ΔPAM: mutated PAM sequence of gRNA. FIG. 2B shows design of homology arms (HAs) surrounding the cut site of guide RNA targeting human TRAC locus. PAM sequence is indicated in the box. FIG. 2B discloses SEQ ID NOS 30 and 29, respectively, in order of appearance. FIG. 2C is a scheme of the construct used for the study. It consists of IL-15 and mClover3 for dual transgene expression; SA: splice acceptor, P2A: “self-cleaving” 2A peptide derived from porcine teschovirus-1, E2A: “self-cleaving” 2A peptide derived from equine rhinitis A virus, bGH poly(A): bovine growth hormone polyadenylation signal.

FIGS. 3A-3C show an overview of the steps to generate transgene knock-in in T cell. FIG. 3A (upper panel) shows molecular steps considering vector design, amplification, purification and concentration DNA. FIG. 3A (middle panel) shows electroporation of RNPs and donor template to T cells using Lonza instrument. FIG. 3A (bottom panel) shows human T cell preparation before electroporation. FIG. 3B is an image of a 1% agarose gel showing PCR amplicons that were gel purified vs. non-gel purified. FIG. 3C is a graph showing concentration of dsDNA template after purification and concentration (n=24).

FIGS. 4A-4D show optimization steps to increase transgene knock-in (KI) efficiency in T cells. FIG. 4A shows knock-in efficiency and cell viability resulted from three different dsDNA template concentrations (n=3 for 1 and 2 μg, n=2 for 3 μg; two-tailed t-test; ns—not significant). FIG. 4B shows knock-in efficiency resulted from two different numbers of T cells used in electroporation (n=5; two-tailed paired t-test, *p=0.041). FIG. 4C shows knock-in efficiency and cell viability resulted from four different lengths of homology arms flanking the transgene of interest (n=2-4; one-way ANOVA; ns—not significant). FIG. 4D shows knock-in efficiency tested at early (4-6 days) and late (>9 days) time points post-electroporation (n=15, two-tailed paired t-test; **p=0.0015).

FIGS. 5A-5B demonstrate validation of transgene expression in gene edited T cells. FIG. 5A is a representative flow graph of GFP expression in gene edited T cells 12 days post electroporation; right panel—overall knock-in efficiency of the transgene after knock-in optimization as determined by flow cytometry of GFP+/TCRαβ− cells (n=7). FIG. 5B shows IL-15 production from gene edited T cells was detected by ELISA 8-10 days post electroporation (n=3, two-tailed t-test, *p=0.024).

FIGS. 6A-6D demonstrate IL-15.E2A.mClover3 knock-in into IL-13 locus. FIG. 6A shows transgene expression evaluated by flow cytometry of GFP+ T cells 10 days post electroporation; left panel—representative flow plots (samples electroporated without template DNA (−DNA) served as controls); right panel—summary graph of left panel (n=8; two-tailed t-test; ***p=0.0004). FIG. 6B shows fold increase of knock-in efficiency without (−) or with (+) T cell activation (n=6; two-tailed t-test; ***p=0.0005). FIG. 6C shows knock-out of IL-13 as confirmed by IL-13 secretion; Ctrl: control knock-out, −DNA: knock-in without DNA template; +DNA: knock-in with DNA template (n=3-11; one-way ANOVA; *p=0.036 **p=0.007). FIG. 6D shows IL-15 expression from IL-13 edited (10 days post-electroporation) T cells 24 hours post T cell activation (n=4, two-tailed t-test; **p=0.005).

FIGS. 7A-7D demonstrate IL-15.E2A.mClover3 transgene integration into TRAC locus. FIG. 7A depicts gating strategy for detection of transgene expression in T cells by flow cytometry. FIG. 7B shows knock-out efficiency of TCR in T cells without (−) or with (+) dsDNA template as measured by flow cytometry (n=5-12). FIG. 7C shows editing of TRAC locus with gRNA (Eyquem et al., 2017) as determined by targeted NGS, n=3. FIG. 7D shows a summary of the most frequent indels by deep sequencing after editing of TCR locus in T cells (sample representative from n=3). The asterisk indicates an unedited allele. FIG. 7D discloses SEQ ID NOS 31-43, respectively, in order of appearance.

FIGS. 8A-8B demonstrate applications of site-specific transgene integration using CRISPR-Cas9. FIG. 8A shows successful knock-in of a CAR and a BiTE into the TRAC locus as confirmed by flow cytometry. FIG. 8B shows that IL-13 production is increased upon T cell activation (−act—non-activated; +act—activated) (n=3).

### DETAILED DESCRIPTION

The present disclosure provides, among other things, methods of genetically modifying an immune cell (e.g., T cell, NK cell) comprising introducing into the immune cell at least one transgene. The transgene is inserted in a genomic locus such that expression of the at least one transgene is under the control of an endogenous promoter. The endogenous promoter is preferably one that can be is inducible by activation of the immune cell. As exemplified in Example 7 below, transgene integration into the IL-13 locus of T cells can create an inducible system controlled by T cell activation.

In some aspects, the present disclosure also provides an optimized protocol for a knock-in strategy using a double-stranded DNA (dsDNA) as a donor DNA template to insert a transgene of interest into a specific region in the genome of the immune cell. For the knock-in experiments, it is demonstrated, as shown in the Examples section below, that non-viral DNA such as dsDNA can be used as a homology-directed repair (HDR) template to achieve efficient knock-in of the transgene. One embodiment of the optimized protocol is detailed in the Examples section (e.g., Example 8) below.

### Definitions

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs” refers to T cells that suppress an abnormal or excessive immune response and play a role in immune tolerance. Tregs cells are typically transcription factor Foxp3-positive CD4+ T cells and can also include transcription factor Foxp3-negative regulatory T cells that are IL-10-producing CD4+ T cells.

The terms “natural killer cell” and “NK cell” are used interchangeable and used synonymously herein. As used herein, NK cell refers to a differentiated lymphocyte with a CD 16+ CD56+ and/or CD57+ TCR− phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

The term “chimeric antigen receptor” or “CAR” as used herein is defined as a cell-surface receptor comprising an extracellular target-binding domain, a transmembrane domain and a cytoplasmic domain, comprising a signaling domain and optionally at least one costimulatory signaling domain, all in a combination that is not naturally found together on a single protein. This particularly includes receptors wherein the extracellular domain and the cytoplasmic domain are not naturally found together on a single receptor protein. The chimeric antigen receptors of the present disclosure are intended primarily for use with lymphocyte such as T cells and natural killer (NK) cells.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding moiety” refers to a target-specific binding element that may be any ligand that binds to the antigen of interest or a polypeptide or fragment thereof, wherein the ligand is either naturally derived or synthetic. Examples of antigen-binding moieties include, but are not limited to, antibodies; polypeptides derived from antibodies, such as, for example, single chain variable fragments (scFv), Fab, Fab′, F(ab′)2, and Fv fragments; polypeptides derived from T Cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. Combinatorial libraries could also be used to identify peptides binding with high affinity to the therapeutic target.

Terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12.

Host cells of the present disclosure include T cells and natural killer cells that contain the DNA or RNA sequences encoding the transgene of interest. Host cells may be used for treatment of cancer, treatment of infection, and/or treatment of autoimmune disease.

The terms “activation” or “stimulation” means to induce a change in their biologic state by which the cells (e.g., T cells and NK cells) express activation markers, produce cytokines, proliferate and/or become cytotoxic to target cells. All these changes can be produced by primary stimulatory signals. Costimulatory signals can amplify the magnitude of the primary signals and suppress cell death following initial stimulation resulting in a more durable activation state and thus a higher cytotoxic capacity. A “costimulatory signal” refers to a signal, which in combination with a primary signal, such as TCR/CD3 ligation, leads to T cell and/or NK cell proliferation and/or upregulation or downregulation of key molecules.

The term “proliferation” refers to an increase in cell division, either symmetric or asymmetric division of cells. The term “expansion” refers to the outcome of cell division and cell death.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of an “exogenous” (i.e., extrinsic or extracellular) nucleic acid into a cell using recombinant DNA technology. The term “genetic modification” means the introduction of an “exogenous” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may be foreign (i.e., not found in the host cell genome) to the host cell, or may be native (i.e., present in the host cell genome) to the cell. The introduced gene or sequence may also be called a “cloned” or “exogenous” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of an exogenous nucleic acid into a cell using a viral vector.

The terms “genetically modified” or “genetically engineered” refers to the addition of extra genetic material in the form of DNA or RNA into a cell.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

The terms “vector”, “cloning vector” and “expression vector” mean the vehicle by which a DNA or RNA sequence (e.g., an exogenous gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Methods of the Disclosure

In one aspect, provided herein is a method of genetically modifying an immune cell, comprising introducing into the immune cell at least one transgene, wherein the at least one transgene is inserted at the interleukin 13 (IL-13) gene locus within the immune cell genome. In some embodiments, at least one transgene is inserted such that expression of the at least one transgene is under the control of the endogenous IL-13 promoter. In some embodiments, expression of the endogenous IL-13 is reduced or abolished.

The IL-13 gene has a cytogenetic location of 5q31.1, and has a molecular location of 132,656,263-132,661,110 (GRCh38/hg38). In some embodiments, the at least one transgene is inserted within or near the above-described location of the IL-13 gene.

In another aspect, provided herein is a method of genetically modifying an immune cell, comprising introducing into the immune cell at least one transgene, wherein the at least one transgene is inserted at the granulocyte-macrophage colony-stimulating factor (GM-CSF) gene locus within the immune cell genome. In some embodiments, at least one transgene is inserted such that expression of the at least one transgene is under the control of the endogenous GM-CSF promoter. In some embodiments, expression of the endogenous GM-CSF is reduced or abolished.

The GM-CSF gene has a cytogenetic location of 5q31.1, and has a molecular location of chr5:132,073,789-132,076,170 (GRCh38/hg38). In some embodiments, the at least one transgene is inserted within or near the above-described location of the GM-CSF gene.

### Transgenes

In various aspects, an exogenous polynucleotide comprising a nucleotide sequence encoding a transgene in introduced into the immune cell. The transgene can be encode any molecule that is beneficial to be expressed in the immune cell, including but not limited to, proteins, peptides and RNAs (e.g., mRNA, siRNA, miRNA).

In some embodiments, at least one transgene encodes a therapeutic molecule. In some embodiments, the therapeutic molecule is selected from a chimeric antigen receptor (CAR), a cytokine, a cytokine receptor, a chimeric cytokine receptor, a switch receptor, a chemokine, an antibody, and a bispecific antibody.

In some embodiments, the therapeutic molecule is a chimeric antigen receptor (CAR). CARs are primarily comprised of 1) an extracellular antigen-binding domain which comprises an antigen-binding moiety, such as a single-chain variable fragment (scFv) derived from an antigen-specific monoclonal antibody, and 2) an intracellular signaling domain, such as the ζ-chain from the T cell receptor CD3. These two regions are fused together via a transmembrane domain. A hinge domain is usually required to provide more flexibility and accessibility between the antigen-binding moiety and the transmembrane domain. Upon transduction, the lymphocyte expresses the CAR on its surface, and upon contact and ligation with the target antigen, it signals through the signaling domain (e.g., CD3ζ chain) inducing cytotoxicity and cellular activation. The CAR may further comprise a leader sequence at the amino-terminus of the extracellular target-binding domain. The leader sequence may be optionally cleaved from the antigen-binding moiety during cellular processing and localization of the CAR to the cellular membrane.

The choice of antigen-binding moiety depends upon the type and number of antigens that define the surface of a target cell. For example, the antigen-binding moiety may be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding moiety that specifically binds to an antigen (e.g., on a cancer cell). Non-limiting examples of cell surface markers that may act as targets for the antigen-binding moiety in the CAR of the invention include those associated with cancer cells. Additionally, the antigen-binding moiety may have recognize an antigen associated with an autoimmune disease, or an infectious disease.

In some embodiments, the antigen-binding moiety comprises an antigen-binding polypeptide or functional variant thereof that binds to an antigen. In some embodiments, the antigen-binding polypeptide is an antibody or an antibody fragment that binds to an antigen. Antigen-binding moieties may comprise antibodies and/or antibody fragments such as monoclonal antibodies, multispecific antibodies, chimeric antibodies, single-chain Fvs (scFv), single chain antibodies, Fab fragments, F(ab′) fragments, disulfide-linked Fvs (sdFv), intrabodies, minibodies, single domain antibody variable domains, nanobodies (VHHs), diabodies and anti-idiotypic (anti-Id) antibodies (including, e.g., anti-Id antibodies to antigen-specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

Exemplary CARs suitable for use in the present disclosure include those that can specifically recognize an antigen such as, but are not limited to, carbonic anhydrase EX, alpha-fetoprotein, A3, antigen specific for A33 antibody, Ba 733, BrE3-antigen, CA125, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, HER2/neu, hypoxia inducible factor (HIF-I), Ia, interleukin 13 receptor α2 (IL13Rα2), insulin growth factor-1 (IGF-I), KC4-antigen, KS-1-antigen, KS1-4, Le-Y, macrophage inhibition factor (MIF), MAGE, MUC1, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

Additional tumor antigens that may be targeted by the CARs described herein include, but are not limited to, a kinase anchor protein 4 (AKAP-4), adrenoceptor beta 3 (ADRB3), anaplastic lymphoma kinase (ALK), immunoglobulin lambda-like polypeptide 1 (IGLL1), androgen receptor, angiopoietin-binding cell surface receptor 2 (Tie 2), B7H3 (CD276), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD38, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLEC12A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (TMPRSS2) ETS fusion gene), ETS translocation-variant gene 6, located on chromosome 12p (ETV6-AML), Fc fragment of IgA receptor (FCAR), Fc receptor-like 5 (FCRL5), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRC5D), ganglioside GD3, ganglioside GM3, glycoceramide (GloboH), Glypican-3 (GPC3), Hepatitis A virus cellular receptor 1 (HAVCR1), hexasaccharide portion of globoH, high molecular weight-melanoma-associated antigen (HMWMAA), human Telomerase reverse transcriptase (hTERT), interleukin 11 receptor alpha (IL-11Ra), KIT (CD117), leukocyte-associated immunoglobulin-like receptor 1 (LAIR1), leukocyte immunoglobulin-like receptor subfamily A member 2 (LILRA2), Lewis(Y) antigen, lymphocyte antigen 6 complex, locus K 9 (LY6K), lymphocyte antigen 75 (LY75), lymphocyte-specific protein tyrosine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAX5), pannexin 3 (PANX3), placenta-specific 1 (PLAC1), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macropain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (TEM1/CD248), tumor endothelial marker 7-related (TEM7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WT1), and X Antigen Family, Member 1A (XAGE1), or a fragment or variant thereof.

In one embodiment, the therapeutic molecule is an IL13Rα2-specific CAR. IL13Rα2, also referred to as CD213A2 (cluster of differentiation 213A2), is a membrane bound protein that in humans is encoded by the IL13RA2 gene.

Further examples of CARs suitable for use in the present disclosure include those that specifically recognize an infectious antigen such as, a viral antigen, a bacterial antigen, a fungal antigen, a parasite antigen, or a prion antigen, or the like. Non-limiting examples of infectious viruses that have been found in humans include but are not limited to: Adenoviridae (most adenoviruses); Arena viridae (hemorrhagic fever viruses); Birnaviridae; Bungaviridae (e.g., Hantaan viruses, bunga viruses, phleboviruses and Nairo viruses); Calciviridae (e.g., strains that cause gastroenteritis); Coronoviridae (e.g., coronaviruses); Filoviridae (e.g., ebola viruses); Flaviridae (e.g., dengue viruses, encephalitis viruses, yellow fever viruses); Hepadnaviridae (Hepatitis B virus); Herpesviridae (herpes simplex virus (HSV) 1 and 2, varicella zoster virus, cytomegalovirus (CMV), herpes virus); Iridoviridae (e.g., African swine fever virus); Norwalk and related viruses, and astroviruses; Orthomyxoviridae (e.g., influenza viruses); Papovaviridae (papilloma viruses, polyoma viruses); Paramyxoviridae (e.g., parainfluenza viruses, mumps virus, measles virus, respiratory syncytial virus); Parvovirida (parvoviruses); Picornaviridae (e.g., polio viruses, hepatitis A virus; enteroviruses, human Coxsackie viruses, rhinoviruses, echoviruses); Poxviridae (variola viruses, vaccinia viruses, pox viruses); Reoviridae (e.g., reoviruses, orbiviruses and rotaviruses); Retroviridae (e.g., human immunodeficiency viruses, such as HIV-1 (also referred to as HTLV-III, LAV or HTLV-III/LAV, or HIV-III); and other isolates, such as HIV-LP); Rhabdoviradae (e.g., vesicular stomatitis viruses, rabies viruses); Togaviridae (e.g., equine encephalitis viruses, rubella viruses); and unclassified viruses (e.g., the etiological agents of Spongiform encephalopathies, the agent of delta hepatitis, the agents of non-A, non-B hepatitis (i.e. Hepatitis C)). Non-limiting examples of infectious bacteria include but are not limited to: Actinomyces israelli, Bacillus antracis, Bacteroides sp., Borrelia burgdorferi, Chlamydia, Clostridium perfringers, Clostridium tetani, Corynebacterium diphtheriae, Corynebacterium sp., Enterobacter aerogenes, Enterococcus sp., Erysipelothrix rhusiopathiae, Fusobacterium nucleatum, Haemophilus influenzae, Helicobacter pyloris, Klebsiella pneumoniae, Legionella pneumophilia, Leptospira, Listeria monocytogenes, Mycobacteria sps. (e.g., M tuberculosis, M avium, M gordonae, M intracellulare, M kansaii), Neisseria gonorrhoeae, Neisseria meningitidis, Pasteurella multocida, pathogenic Campylobacter sp., Rickettsia, Staphylococcus aureus, Streptobacillus monihformis, Streptococcus (anaerobic sps.), Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Treponema pallidium, and Treponema pertenue. Non-limiting examples of infectious fungi include: Cryptococcus neoformans, Histoplasma capsulatuin, Coccidioides immitis, Blastomyces dernatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma. Other medically relevant microorganisms have been descried extensively in the literature, e.g., see C. G. A. Thomas, “Medical Microbiology”, Bailliere Tindall, Great Britain 1983, which is hereby incorporated by reference in its entirety.

Additional examples of CARs suitable for use in the present disclosure include those that specifically recognize an autoimmune antigen such as the antigens associated with e.g., systemic lupus erythematosus, Wegener's granulomatosis, autoimmune hepatitis, Crohn's disease, scleroderma, ulcerative colitis, Sjögren's syndrome, Type 1 diabetes mellitus, uveitis, myocarditis, rheumatic fever, ankylosing spondylitis, rheumatoid arthritis, multiple sclerosis, and psoriasis.

The transmembrane domain in CARs of the present disclosure may be derived from the protein contributing to the extracellular target-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid-binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this invention may be derived from (i.e. comprise at least the transmembrane region(s) of) the α, β or ζ chain of the T-cell receptor, CD28, CD3 epsilon, CD45, CD4, CD5, CD8, CD9, CD16, CD22, CD33, CD37, CD40, CD64, CD80, CD86, CD134, CD137, CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In certain embodiments, the CAR may further comprise a linker region between the extracellular antigen-binding domain and the transmembrane domain, wherein the antigen-binding moiety, linker, and the transmembrane domain are in frame with each other. The term “linker region” as used herein generally means any oligo- or polypeptide that functions to link the antigen-binding moiety to the transmembrane domain. A linker region can be used to provide more flexibility and accessibility for the antigen-binding moiety. A linker region may comprise up to 300 amino acids, preferably 10 to 100 amino acids and most preferably 25 to 50 amino acids. A linker region may be derived from all or part of naturally occurring molecules, such as from all or part of the extracellular region of CD8, CD4 or CD28, or from all or part of an antibody constant region. Alternatively, the linker region may be a synthetic sequence that corresponds to a naturally occurring linker region sequence, or may be an entirely synthetic linker region sequence. Non-limiting examples of linker regions which may be used in accordance to the invention include a part of human CD8a chain, partial extracellular domain of CD28, FcyRllla receptor, IgG, IgM, IgA, IgD, IgE, an Ig hinge, or functional fragment thereof. In some embodiments, additional linking amino acids are added to the linker region to ensure that the antigen-binding moiety is an optimal distance from the transmembrane domain. In some embodiments, when the linker is derived from an Ig, the linker may be mutated to prevent Fc receptor binding.

In some embodiments, the linker domain comprises a hinge domain. The hinge domain may be derived from CD8α, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

CARs of the present disclosure may comprise a cytoplasmic signaling domain, which comprises one or more costimulatory domains and one or more signaling domains. The cytoplasmic domain, which comprises one or more costimulatory domains and one or more signaling domains, is responsible for activation of at least one of the normal effector functions of the lymphocyte in which the CAR has been placed in. The term “effector function” refers to a specialized function of a cell. Effector function of a T cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of costimulatory domains which can be used in the CARs of the present disclosure include, those derived from 4-1BB (CD137), CD28, ICOS, CD134 (OX-40), BTLA, CD27, CD30, GITR, CD226, and HVEM. In some embodiments, the CAR of the present disclosure comprises one costimulatory domain. In some embodiments, the CAR of the present disclosure comprises a costimulatory domain derived from 4-1BB. In some embodiments, the CAR of the present disclosure comprises a costimulatory domain derived from CD28. In some embodiments, the CAR of the present disclosure comprises two costimulatory domains. In some embodiments, the CAR of the present disclosure comprises a costimulatory domain derived from 4-1BB and a costimulatory domain derived from CD28.

Non-limiting examples of signaling domains which can be used in the CARs of the present disclosure include, e.g., signaling domains derived from DAP10, DAP12, Fc epsilon receptor I gamma chain (FCER1G), FcR β, CD3δ, CD3ε, CD3γ, CD3ζ, CD5, CD22, CD226, CD66d, CD79A, and CD79B. In some embodiments, the CAR of the present disclosure comprises a signaling domain derived from CD3ζ.

In some embodiments, the signaling domain(s) and costimulatory domain(s) can be in any order. In some embodiments, the signaling domain is upstream of the co-stimulatory domains. In some embodiments, the signaling domain is downstream from the costimulatory domains. In the cases where two or more costimulatory domains are included, the order of the costimulatory domains could be switched.

In certain embodiments, CARs of the present disclosure may be regulated by a safety switch. As used herein, the term “safety switch” refers to any mechanism that is capable of removing or inhibiting the effect of a CAR from a system (e.g., a culture or a subject). Safety switches can function to increase the safety of the CAR.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105(11): 4247-4254; US Publ. No. 2011/0286980, each of which are incorporated herein by reference in their entirety for all purposes).

CARs of the present disclosure may further comprise an accessory gene that encodes an accessory peptide. Examples of accessory genes can include a transduced host cell selection marker, an in vivo tracking marker, a cytokine, a suicide gene, or some other functional gene. In certain embodiments, the functional accessory gene can increase the safety of the CAR. In certain embodiments, the CAR comprises at least one accessory gene. In certain embodiments, the CAR comprises one accessory gene. In other embodiments, the CAR comprises two accessory genes. In yet another embodiment, the CAR comprises three accessory genes. For example, the CAR construct may comprise an accessory gene which is truncated CD19 (tCD19). The tCD19 can be used as a tag. Expression of tCD19 may also help determine transduction efficiency.

In some embodiments, the therapeutic molecule is a cytokine. The cytokines may be a secretable cytokine (e.g., but not limited to, IL-7, IL-12, IL-15, IL-18) or a membrane bound cytokine (e.g., but not limited to, IL-15). Additional examples of cytokines include, but are not limited to, interferons (e.g., IFN-γ, IFN-α, IFN-β, IFN-ω, IFN-τ), interleukins (e.g., IL-1, IL-2, including, e.g., Proleukin®, IL-3, IL-4, IL-5, IL-6, IL-7, IL-8, IL-9), and transforming growth factors (e.g., TGF-β1, TGF-β2 and TGF-β3). In one embodiment, the therapeutic molecule is IL-15.

In some embodiments, the therapeutic molecule is a cytokine receptor. The cytokine receptor may be a natural cytokine receptor (e.g., an interleukin receptor), or a chimeric cytokine receptor or a switch receptor (e.g., but not limited to, IL-2/IL-7, IL-4/IL-7), a constitutive active cytokine receptor (e.g., but not limited to, C7R), or a dominant negative receptors (DNR; e.g., but not limited to TGFRII DNR).

In some embodiments, the therapeutic molecule is a chemokine. Examples of chemokines include, but not limited to, IP-10, Mig, Groa/IL-8, RANTES, MIP-1α, MIP-1β, MCP-1, PF-4, and the like.

In some embodiments, the therapeutic molecule is an antibody. Suitable antibodies include, but are not limited to, monoclonal antibodies such as Rituxan® (rituximab), Remicade® (infliximab), Herceptin® (trastuzumab), Humira™ (adalimumab), Xolair® (omalizumab), Bexxar® (tositumomab), Raptiva™ (efalizumab), Erbitux™ (cetuximab), and the like, or bispecific antibodies such as bispecific T-cell engagers (BiTEs), or an antibody fragment (e.g., antigen-binding fragment of a monoclonal antibody, a single chain variable fragment (scFv), a Fab, a Fab′, a F(ab′)2, and a Fv fragment) thereof. In one embodiment, the therapeutic molecule is a BiTE that binds specifically to Tumor Endothelial Marker 8 (TEM8) and CD3.

Further classes of transgenes that can be introduced to the immune cells, include engineered T cell receptors (TCRs), and ligands of costimulatory molecules (e.g., but not limited to, CD80, 4-1BBL).

It is contemplated that multiple transgenes can be introduced to the immune cell using the methods of the present disclosure. For example, two, three, four, five, six, seven, eight, nine, ten or more transgenes may be introduced to the immune cell. The multiple transgenes may be introduced in one nucleotide sequence or may be introduced in separate nucleotide sequences. The multiple transgenes may be inserted into the same genomic locus (e.g., IL-13 locus, or GM-CSF locus) in the immune cell or may be inserted into different genomic loci (e.g., a subset is inserted into IL-13 locus and another subset is inserted into GM-CSF locus) in the immune cell. The multiple transgenes may be introduced into the immune cell in one genetic modification step or in several genetic modification steps. The multiple transgenes may be independently selected from those described above.

In various embodiments, the nucleotide sequence comprising the at least one transgene is operably linked to at least a regulatory element. The regulatory element can be capable of mediating expression of the transgene in the host cell. Regulatory elements include, but are not limited to, promoters, enhancers, initiation sites, polyadenylation (polyA) tails, Internal Ribosome Entry Site (IRES) elements, response elements, and termination signals. In certain embodiments, the regulatory element regulates transgene expression. In certain embodiments, the regulatory element increases the expression of the transgene. In certain embodiments, the regulatory element increases the expression of the transgene once the host cell is activated. In certain embodiments, the regulatory element decreases expression of the transgene. In certain embodiments, the regulatory element decreases expression of the transgene once the host cell is activated.

In various embodiments, the nucleotide sequence comprising the at least one transgene also comprises a sequence encoding a self-cleaving peptide and/or an internal ribosomal entry site (IRES). In some embodiments, the sequence encoding a self-cleaving peptide and/or an IRES is located 5′ and/or 3′ to the transgene to allow cleavage of the transgene from the endogenous gene. In some embodiments, the sequence encoding a self-cleaving peptide and/or IRES is located 5′ to the transgene. In some embodiments, when two or more transgenes are encoded, the transgenes may be separated by a sequence encoding a self-cleaving peptide and/or an IRES.

In some embodiments, the self-cleaving peptide is a 2A sequence. Non-limiting examples of 2A sequences includes Thoseaasigna virus 2A (T2A; EGRGSLLTCGDVEENPGP, SEQ ID NO: 18 or GSGEGRGSLLTCGDVEENPGP, SEQ ID NO: 19); the foot and mouth disease virus (FMDV) 2A sequence (F2A; GSGSRVTELLYRMKRAETYCPRPLLAIHPTEARHKQKIVAPVKQLLNFDLLKLAGDVESNPGP, SEQ ID NO: 20), Sponge (Amphimedon queenslandica) 2A sequence (LLCFLLLLLSGDVELNPGP, SEQ ID NO: 21; or HHFMFLLLLLAGDIELNPGP, SEQ ID NO: 22); acorn worm 2A sequence (Saccoglossus kowalevskii) (WFLVLLSFILSGDIEVNPGP, SEQ ID NO: 23); amphioxus (Branchiostoma floridae) 2A sequence (KNCAMYMLLLSGDVETNPGP, SEQ ID NO: 24; or MVISQLMLKLAGDVEENPGP, SEQ ID NO: 25); porcine teschovirus-1 2A sequence (P2A; GSGATNFSLLKQAGDVEENPGP, SEQ ID NO: 26); and equine rhinitis A virus 2A sequence (E2A; GSGQCTNYALLKLAGDVESNPGP, SEQ ID NO: 27). In some embodiments, the separation sequence is a naturally occurring or synthetic sequence. In certain embodiments, the separation sequence includes the 2A consensus sequence D-X-E-X-NPGP (SEQ ID NO: 28), in which X is any amino acid residue.

Alternatively, an Internal Ribosome Entry Site (IRES) may be used. IRES is an RNA element that allows for translation initiation in a cap-independent manner. IRES can link two coding sequences in one nucleotide sequence and allow the translation of both molecules in cells.

In various embodiments, the nucleotide sequence comprising the at least one transgene also comprises a polyadenylation (polyA) sequence. The polyA sequence is typically included at the 3′ end of a coding sequence and is part of the process that produces mature messenger RNA (mRNA) for translation.

In various embodiments, the at least one transgene may be operatively linked to at least one insulator and/or enhancer sequence. Enhancers are a short region of DNA that can increase transcription of genes. Enhancer sequence can be identified by screening for the presence of “enhancer locators”, such as p300, histone methylation marks (e.g., H3K4mel⁺) or acetylation marks (e.g., H3K27ac⁺). Certain promoters such as the human cytomegalovirus promoter can also serve as enhancers. Examples of insulator sequences include, but are not limited to, those derived from CTCF-binding site, β-globin loci, and TFIIIC-binding site.

### Genome Editing Techniques

In various embodiments, immune cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. The term “site-specific nuclease” as used herein refers to a nuclease capable of specifically recognizing and cleaving a nucleic acid (DNA or RNA) sequence. Suitable site-specific nucleases for use in the present invention include, but are not limited to, RNA-guided endonucleases (e.g., CRISPR-associated (Cas) proteins), zinc finger nucleases, TALEN nucleases, meganucleases, or mega-TALEN nucleases. For example, a site-specific nuclease (e.g., a Cas9+ guide RNA) capable of inducing a double strand break in a target DNA sequence is introduced to a host cell, along with a donor polynucleotide encoding a transgene of interest.

In some embodiments, the site-specific nuclease comprises a Cas protein and a guide RNA (gRNA) which specifically binds to the target locus. The terms “guide RNA,” “guide RNA molecule,” “gRNA molecule” or “gRNA” are used interchangeably, and refer to a set of nucleic acid molecules that promote the specific directing of a RNA-guided endonuclease or other effector molecule (typically in complex with the gRNA molecule) to a target sequence. In some embodiments, the directing is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In some embodiments, a gRNA molecule consists of a single contiguous polynucleotide molecule, referred to herein as a “single guide RNA” or “sgRNA”. In other embodiments, a gRNA molecule consists of a plurality, usually two, polynucleotide molecules, which are themselves capable of association, usually through hybridization. For CRISPR-Cas9 systems, gRNA molecules generally include a targeting domain and a trans-activating CRISPR RNA (tracrRNA). In some embodiments the targeting domain and tracrRNA are disposed on a single polynucleotide. For Cas9, for example, a single-guide RNA can comprise a crRNA fused to a tracrRNA (e.g., via a linker). In other embodiments, the targeting domain and tracrRNA are disposed on separate polynucleotides.

Examples of Cas proteins useful in the methods of the present disclosure include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In some embodiments, the Cas protein used in the methods described herein is a Cas9 protein. The Cas9 protein may be from S. pyogenes, Streptococcus thermophilus, Neisseria meningitidis, F. novicida, S. mutans or Treponema denticola.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity.

In some embodiments, the guide RNA comprises a nucleotide sequence which is complementary to a sequence in the IL-13 gene. In one embodiment, the guide RNA comprises a nucleotide sequence of GAGGAGCGGAUGCAUAGGCU (SEQ ID NO: 16) or GGAUUGAGGAGCGGAUGCAU (SEQ ID NO: 17), or a nucleotide sequence having at least at least 80, at least 85, at least 90, or at least 95% sequence identity thereof of either.

In some embodiments, the guide RNA comprises a nucleotide sequence which is complementary to a sequence in the GM-CSF gene.

In particular embodiments, the RNA-guided endonuclease is introduced as a ribonucleoprotein (RNP) complex comprising the gRNA and a Cas protein (e.g., Cas9). In certain embodiments, the RNP is introduced via electroporation, particle gun, calcium phosphate transfection, cell compression or squeezing. In some embodiments, the RNP is introduced via electroporation. In some embodiments, the RNA-guided endonuclease is introduced as one or more polynucleotide encoding the gRNA and/or a Cas protein. For example, the RNA-guided endonuclease introduced as an mRNA encoding the Cas protein and a vector (e.g., AAV) encoding the gRNA. As another example, the RNA-guided endonuclease can be introduced as a vector (e.g., AAV) encoding both the gRNA and/or the Cas protein.

In some embodiments where the RNA-guided endonuclease is delivered as a ribonucleoprotein (RNP) complex, one or more polymers may be added to increase the stability of RNPs. For example, polyglutamic acid (PGA) may be added which can stabilize the RNPs, resulting in an increased editing efficiency while sustaining T cell viability. See e.g., Nguyen et al., Nat Biotechnol, 2020. 38(1): p. 44-49, which is incorporated herein by reference in its entirety.

In alternative embodiments, the site-specific nuclease used in the methods described herein is a zinc finger nuclease, a transcription activator-like effector nuclease (TALEN), a mega-TALEN nuclease, a meganuclease and/or a restriction endonuclease.

The site-specific nuclease used in the methods described herein may include a zinc finger nuclease (ZFN). Zinc finger nucleases (ZFNs) are a class of engineered DNA-binding proteins that assist targeted editing of the genome by creating double-strand breaks in DNA at targeted locations. ZFNs typically comprise two functional domains: a) a DNA-binding domain comprising a chain of two-finger modules (each recognizing a unique hexamer (6 bp) sequence of DNA-two-finger modules are stitched together to form a Zinc Finger Protein, each with specificity of about 24 bp or more) and b) a DNA-cleaving domain comprising the nuclease domain of Fok I. When the DNA-binding and -cleaving domains are fused together, a ZFN can act like a highly-specific pair of “genomic scissors”.

The site-specific nuclease used in the methods described herein may include a transcription activator-like effector nuclease (TALEN). Transcription activator-like effector nucleases (TALEN) are a class of sequence-specific nucleases that can be used to make double-strand breaks at specific target sequences in the genome of a prokaryotic or eukaryotic organism. They typically comprise a TAL effector DNA-binding domain fused to a DNA cleavage domain (a nuclease which cuts DNA strands). TAL effector nucleases can be created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. doi: 10.1093/nar/gkg704; and Miller et al. (2011) Nature Biotechnology 29:143-148; all of which are herein incorporated by reference in their entirety and for all purposes.

In various embodiments, the at least one transgene is introduced into the immune cell via a donor polynucleotide. Polynucleotide transfer may be via viral or non-viral gene methods. Suitable methods for polynucleotide delivery for use with the current methods include any method known by those of skill in the art, by which a polynucleotide can be introduced into an organelle, cell, tissue or organism.

In some embodiments, the donor polynucleotide is a non-viral polynucleotide. For example, the donor polynucleotide can be a single-stranded DNA (ssDNA), a double-stranded DNA (dsDNA), a plasmid, or a transposon (such as a PiggyBac- or a Sleeping Beauty transposon).

In some embodiments, the donor polynucleotide is a double-stranded DNA (dsDNA).

In some embodiments, the donor polynucleotide is a single-stranded DNA (ssDNA). Although not wishing to be bound by theory, ssDNA may trigger a different repair pathway and lead to improved knock-in efficiency and potentially less non-specific integration [23, 24]. Commercially available kits may be employed for fast and efficient ssDNA generation.

In alternative embodiments, the donor polynucleotide is included in a viral vector. The viral vector may be a retroviral vector, an adenoviral vector, an adeno-associated virus vector, an alphaviral vector, a herpes virus vector, and a vaccinia virus vector.

In various embodiments, the donor polynucleotide comprises the structure [5′ homology arm]-[at least one transgene]-[3′ homology arm]. In certain embodiments, the 5′ homology arm and 3′ homology arm comprises nucleic acid sequences homologous to nucleic acid sequences flanking the at least one target site. In some embodiments, the 5′ homology arm comprises nucleic acid sequences that are homologous to nucleic acid sequences 5′ of the target site. In some embodiments, the 3′ homology arm comprises nucleic acid sequences that are homologous to nucleic acid sequences 3′ of the target site.

In some embodiments, the 5′ homology arm and a 3′ homology arm independently have between about 100 to about 1500 base pairs (bp) in length. In some embodiments, the 5′ homology arm and the 3′ homology arm independently have from about 100 bp to 200 bp, 100 bp to 400 bp, 200 bp to 400 bp, 200 bp to 500 bp, 300 bp to 600 bp, 400 bp to 800 bp, 500 bp to 900 bp, 600 bp to 1000 bp, 800 bp to 1200 bp, or 1000 bp to 1500 bp in length. In some embodiments, the 5′ homology arm and the 3′ homology arm independently have about 100 bp, about 150 bp, about 200 bp, about 250 bp, about 300 bp, about 350 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1000 bp, about 1050 bp, about 1100 bp, about 1150 bp, about 1200 bp, about 1250 bp, about 1300 bp, about 1350 bp, about 1400 bp, about 1450 bp, or about 1500 bp in length. In some embodiments, The 5′ homology arm and the 3′ homology arm have identical length. In some embodiments, The 5′ homology arm and the 3′ homology arm have different length.

In some embodiments, one or more truncated target sequences of the site-specific nuclease may be incorporated at the ends of homology arms in the donor polynucleotide. Although not wishing to be bound by theory, the truncated target sequences interact with site-specific nuclease to shuttle the donor polynucleotide to the nucleus, thereby enhancing HDR efficiency. See e.g., Nguyen et al., Nat Biotechnol, 2020. 38(1): p. 44-49, which is incorporated herein by reference in its entirety.

In some embodiments, the 5′ homology arm in the donor polynucleotide comprises the nucleotide sequence of SEQ ID NO: 14, or a nucleotide sequence having at least 70, at least 75, at least 80, at least 85, at least 90, at least 95, at least 96, at least 97, at least 98 or at least 99% identity thereof, or a fragment thereof.

In some embodiments, the 3′ homology arm in the donor polynucleotide comprises the nucleotide sequence of SEQ ID NO: 15, or a nucleotide sequence having at least 70, at least 75, at least 80, at least 85, at least 90, at least 95, at least 96, at least 97, at least 98 or at least 99% identity thereof, or a fragment thereof.

In various embodiments, the site-specific nuclease and/or the donor polynucleotide is introduced to the immune cell via a physical means. For example, the physical means may be electroporation, microinjection, magnetofection, ultrasound, a ballistic or hydrodynamic method, or a combination thereof.

Electroporation is a method of polynucleotide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues). intravenous, intraprotatic, intratumor, intralymphatic (allows direct administration of DCs) and intraperitoneal. It is understood that proper site of injection preparation is necessary (e.g., shaving of the site of injection to observe proper needle placement).

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In some embodiments, the site-specific nuclease and the donor polynucleotide are both introduced to the immune cell via electroporation. In some embodiments, the electroporation is conducted by mixing about 0.25×10⁶-2.0×10⁶ immune cells with about 1 μg-3 μg of the donor polynucleotide (e.g., dsDNA). In one embodiment, the electroporation is conducted by mixing about 1.0×10⁶ immune cells with about 2 μg of the donor polynucleotide (e.g., dsDNA).

Nucleic acid vaccines can be used to transfer donor polynucleotides into the immune cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the immune cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993).

The genetically modifying step may be conducted ex vivo or in vivo. In some embodiments, the genetically modifying step is conducted ex vivo. The method may further include activation and/or expansion of the host cell ex vivo before, after and/or during the genetic modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity. In certain embodiments, the host cells may be removed from the subject and modified ex vivo using the nucleases and/or polynucleotides (e.g., donor polynucleotide) of the invention. In certain embodiments, the host cells obtained from the subject can be modified ex vivo using the nucleases and/or polynucleotides (e.g., donor polynucleotide) of the invention and then administered back to the subject.

In certain embodiments, the immune cells are genetically modified to express a transgene described above. In some embodiments, the immune cells are genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours.

In some embodiments, the immune cell is allowed to recover for about 4-12 days after introduction of the at least one transgene. In some embodiments, the immune cell is allowed to recover for about 4, 5, 6, 7, 8, 9, 10, 11, and 12 days after introduction of the at least one transgene. In some embodiments, the immune cell is allowed to recover more than 9 days after introduction of the at least one transgene.

In various embodiments, the immune cell is a T cell, a natural killer (NK) cell, a mesenchymal stem cell (MSC), or a macrophage.

In various embodiments, the immune cell is a T cell. T cells may include, but are not limited to, thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+ CD8+ T cell, or any other subset of T cells. Other illustrative populations of T cells suitable for use in particular embodiments include naive T cells memory T cells, and NKT cells.

In some embodiments, the T cell is a CD8+ T cell, a CD4+ T cell, a cytotoxic T cell, an αβ T cell receptor (TCR) T cell, a natural killer T (NKT) cell, a γδ T cell, a memory T cell, a T-helper cell, a regulatory T cell (Treg), an invariant natural killer T (iNKT) cell, a memory T-cell, a memory stem T-cell (TSCM), a naïve T-cell, and/or an effector T-cell.

In various embodiments, the immune cell is a NK cell. NK cell refers to a differentiated lymphocyte with a CD3− CD16+, CD3− CD56+, CD16+ CD56+ and/or CD57+ TCR− phenotype.

In various embodiments, the immune cell has been activated and/or expanded ex vivo.

In some embodiments, the immune cell is derived from a blood, marrow, tissue, or a tumor sample.

### Isolation/Enrichment

The immune cells may be autologous/autogeneic (“self”) or non-autologous (“non-self,” e.g., allogeneic, syngeneic or xenogeneic) with respect to the subject receiving the cells. In certain embodiments, the immune cells are obtained from a mammalian subject. In other embodiments, the immune cells are obtained from a primate subject. In certain embodiments, the immune cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, immune cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof using either positive or negative selection techniques. In certain embodiments, the T lymphocytes for use in the compositions of the invention do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

### Stimulation/Activation

In various embodiments, the method of genetic modification further includes a step of activating the immune cell. According to the methods of the present disclosure, expression of the at least one transgene is increased after the cell is activated.

In order to reach sufficient therapeutic doses of immune cell compositions, immune cells are often subjected to one or more rounds of stimulation/activation. In certain embodiments, a method of producing immune cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing immune cells for administration to a subject comprises stimulating the immune cells to become activated and to proliferate in the presence of one or more stimulatory signals or agents.

Immune cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the T cells can be activated by binding to an agent that activates CD3ζ.

In certain embodiments, the T cells are activated by CD3, CD28 and/or CD2 stimulation.

In other embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used.

In certain embodiments, the immune cells are activated by administering phorbol myristate acetate (PMA) and ionomycine. In certain embodiments, the immune cells are activated by administering an appropriate antigen that induces activation and then expansion. In certain embodiments, PMA, ionomycin, and/or appropriate antigen are administered with CD3 induce activation and/or expansion.

In general, the activating agents used in the present invention includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD3ζ agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 μg/ml. In certain embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the CD3-binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety.

In certain embodiments, the NK cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In certain embodiments, the NK cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include but are not limited to interferon alpha and interferon gamma, and such cytokines include but are not limited to IL-15, IL-2, IL-21.

In certain embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Expansion/Proliferation

After the immune cells are activated and transduced, the cells are cultured to proliferate. Immune cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120). Other illustrative examples for agents that may be used for the expansion of T cells are agents that bind to CD8, CD45 or CD90, such as αCD8, αCD45 or αCD90 antibodies. Illustrative examples of T cell population including antigen-specific T cells, T helper cells, cytotoxic T cells, memory T cell (an illustrative example of memory T-cells are CD62L|CD8| specific central memory T cells) or regulatory T cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

After the immune cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In certain embodiments, the binding agent includes antibodies (see for example Hoshino et al, Blood. 1991 Dec. 15; 78(12):3232-40). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for immune cell expansion include, but are not limited to, surfactant, plasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

### Compositions of the Disclosure

Compositions of the present disclosure include, but are not limited to, genetically modified immune cells, and pharmaceutical compositions comprising the genetically modified immune cells.

In some aspects, the present disclosure provides genetically modified immune cells prepared according to the methods described herein.

In one aspect, provided herein is a genetically modified immune cell, comprising at least one transgene inserted at the IL-13 gene locus within the immune cell genome. In some embodiments, expression of the at least one transgene is under the control of the endogenous IL-13 promoter. In some embodiments, expression of the endogenous IL-13 is reduced or abolished.

In one aspect, provided herein is a genetically modified immune cell, comprising at least one transgene inserted at the GM-CSF gene locus within the immune cell genome. In some embodiments, expression of the at least one transgene is under the control of the endogenous GM-CSF promoter. In some embodiments, expression of the endogenous GM-CSF is reduced or abolished.

In various embodiments, the immune cell is a T cell, a natural killer (NK) cell, a mesenchymal stem cell (MSC), or a macrophage.

In various embodiments, the immune cell is a T cell. T cells may include, but are not limited to, thymocytes, naive T lymphocytes, immature T lymphocytes, mature T lymphocytes, resting T lymphocytes, or activated T lymphocytes. A T cell can be a T helper (Th) cell, for example a T helper 1 (Thl) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+ CD8+ T cell, or any other subset of T cells. Other illustrative populations of T cells suitable for use in particular embodiments include naive T cells memory T cells, and NKT cells.

In some embodiments, the T cell is a CD8+ T cell, a CD4+ T cell, a cytotoxic T cell, an αβ T cell receptor (TCR) T cell, a natural killer T (NKT) cell, a γδ T cell, a memory T cell, a T-helper cell, a regulatory T cell (Treg), an invariant natural killer T (iNKT) cell, a memory T-cell, a memory stem T-cell (TSCM), a naïve T-cell, and/or an effector T-cell.

In various embodiments, the immune cell is a NK cell. NK cell refers to a differentiated lymphocyte with a CD3− CD16+, CD3− CD56+, CD16+ CD56+ and/or CD57+ TCR− phenotype.

In various embodiments, the immune cell has been activated and/or expanded ex vivo.

In some embodiments, the immune cell is derived from a blood, marrow, tissue, or a tumor sample.

In one aspect, provided herein is a pharmaceutical composition comprising the genetically modified immune cell described herein, and a pharmaceutically acceptable carrier.

In some embodiments, the compositions comprise one or more polypeptides of the CARs and other related molecules (e.g., CD20 or anti-CD20 antibody), polynucleotides, vectors comprising same, and cell compositions, as disclosed herein. Compositions of the present disclosure include, but are not limited to pharmaceutical compositions.

In one aspect, the present disclosure provides a pharmaceutical composition comprising a polynucleotide or a recombinant vector described herein, and a pharmaceutically accepted carrier and/or excipient.

Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising genetically modified immune cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising genetically modified immune cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the genetically modified immune cells may be mixed with substances that adhere or penetrate then prior to their administration, e.g., but not limited to, nanoparticles.

### Therapeutic Methods

In some aspects, the present disclosure provides a method of treating a disease in a subject in need thereof. A therapeutically effective amount of the genetically modified immune cells described herein or the pharmaceutical composition comprising the immune cells is administered to the subject. In some embodiments, the disease is a cancer, an autoimmune disease, or an infectious disease.

In some embodiments, the therapeutic method of the present disclosure includes one or more of the following steps:
a) isolating T cells and/or NK cells from the subject;
b) genetically modifying said T cells and/or NK cells ex vivo by introducing into the T cells and/or NK cells at least one transgene, wherein the at least one transgene is inserted at the IL-13 gene locus or GM-CSF gene locus within the genome of the T cells and/or NK cells;
c) optionally, expanding and/or activating said T cells and/or NK cells before, after or during step (b); and
d) introducing the genetically modified T cells and/or NK cells into the subject.

In some embodiment, the disease is a cancer. The terms “cancer” and “cancerous” refer to or describe the physiological condition in mammals that is typically characterized by unregulated cell growth. The term “cancer” includes, for example, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of cancer can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, immune cells modified with an IL13Rα2-binding CAR, or pharmaceutical compositions thereof, are administered to a subject to treat a cancer expressing IL13Rα2. The IL13Rα2+ cancer may be a brain cancer such as glioblastoma, a colon cancer, a renal cell carcinoma, a pancreatic cancer, a melanoma, a head and neck cancer, a mesothelioma, or an ovarian cancer.

In some embodiment, the compositions and methods described in the present disclosure are used to treat an autoimmune disease. Non-limiting examples of autoimmune diseases that may be treated with the compositions and methods described herein include but are not limited to systemic lupus erythematosus, Wegener's granulomatosis, autoimmune hepatitis, Crohn's disease, scleroderma, ulcerative colitis, Sjögren's syndrome, Type 1 diabetes mellitus, uveitis, myocarditis, rheumatic fever, ankylosing spondylitis, rheumatoid arthritis, multiple sclerosis, and psoriasis.

In some embodiment, the compositions and methods described in the present disclosure are used to treat an infectious disease. Infectious diseases are well known to those skilled in the art, and non-limiting examples include but are not limited to infections of viral etiology such as HIV, influenza, Herpes, viral hepatitis, Epstein Bar, polio, viral encephalitis, measles, chicken pox, Papilloma virus; infections of bacterial etiology such as pneumonia, tuberculosis, syphilis; or infections of parasitic etiology such as malaria, trypanosomiasis, leishmaniasis, trichomoniasis, amoebiasis.

In some embodiments, the modified immune cell is an autologous cell. In some embodiments, the modified immune cell is an allogeneic cell. In cases where the immune cell is isolated from a donor, the method may further include a method to prevent graft vs host disease (GVHD) and the immune cell rejection.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified immune cells. It is also contemplated that a variety of doses will be effective to improve in vivo effector function of modified immune cells.

In some embodiments, composition comprising the modified immune cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of modified immune cells will depend on the therapeutic use for which the composition is intended for.

Modified immune cells may be administered multiple times at dosages listed above. The modified immune cells may be allogeneic, syngeneic, xenogeneic, or autologous to the patient undergoing therapy.

The compositions and methods described in the present disclosure may be utilized in conjunction with other types of therapy for cancer, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

As a non-limiting example, the invention can be combined with other therapies that block inflammation (e.g., via blockage of IL1, INFα/β, IL6, TNF, IL23, etc.).

The methods and compositions of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the invention can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the invention can be combined with additional immunotherapies and therapies. For example, when used for treating cancer, the compositions of the invention can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified immune cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Materials and Methods

The following materials and methods were used in Examples 1-7.

Generation of dsDNA Donor Template

Construct Design

All construct for the gene insertion were designed using SnapGene™ and then synthesized by GeneArt/Life Technologies Corporation (ThermoFisher), and subcloned into pMA vector as a final product. Detail description of IL-15.E2A.mClover3 construct is provided in the Results section. IL13Rα2-specific CAR sequence has been previously described [31]. The BiTE sequence is as follow: TEM8-specific scFv L2 [32, 33], a short serine-glycine linker and CD3-specific scFV [34] followed by 2A sequence and Q8 tag for detection. Both constructs contain HAs for TRAC locus.

PCR Amplification

Plasmids were transformed and amplified in DH5a bacterial cells and grown overnight. DNA was extracted by Nucleobond Endotoxin-free Maxiprep (Takara Bio). Primers were designed using SnapGene™ for different homology arm lengths (100, 200, 300 and 400 bp) for insertion in the TRAC locus (Table 1). Rules used for primer design were: ±50% CG content, less than 22 bp in length, and melting temperatures below 60° C. The dsDNA was generated by PCR amplification using CloneAmp HiFi Taq polymerase (Takara Bio), forward and reverse primer (0.5 μM), plasmid DNA (15-20 ng), and nuclease-free water, in a final volume of 50 μL. Reactions were run on the ProFlex™ PCR System (ThermoFisher) according to the following program: initial denaturation at 98° C. for 30 seconds, 20 cycles of each 3 steps (denaturation at 98° C. for 10 seconds, annealing at +3° C. of lower melting temperature of primer for 15 seconds, and extension at 72° C. for variable time based on PCR product size—5 sec/kb), final extension at 72° C. for 3 minutes. Two PCR reactions combined were run on 1% agarose gel for band size confirmation. The BenchTop 1 kb DNA Ladder (Promega) was used in all experiments. To generate highly concentrated dsDNA, 8 PCR reactions were run in total and 2 of these reactions were combined in one gel slot.

TABLE 1
Primer sequences

| Primer | Primer sequence (5′-3′) | Tm (° C.) |
|---|---|---|
| TRAC 100 HA | F: CTTGTCCATCACTGGCATCTG (SEQ ID NO: 1) | 55.9 |
| | R: CGGTGAATAGGCAGACAGAC (SEQ ID NO: 2) | 55.2 |
| TRAC 200 HA | F: GCCAAGATTGATAGCTTGTGC (SEQ ID NO: 3) | 54.5 |
| | R: GTCAGATTTGTTGCTCCAGGC (SEQ ID NO: 4) | 56.6 |
| TRAC 300 HA | F: GCAGTATTATTAAGTAGCCCTG (SEQ ID NO: 5) | 50.7 |
| | R: CGAAGGCACCAAAGCTG (SEQ ID NO: 6) | 54.3 |
| TRAC 400 HA | F: CAGTTTGCTTTGCTGGGCCTT (SEQ ID NO: 7) | 59.1 |
| | R: GGCAATGGATAAGGCCGA (SEQ ID NO: 8) | 55.2 |

DNA Purification and Concentration

The amplicons with the size corresponding transgene DNA size were excised from the gel and purified using the NucleoSpin® Gel and PCR clean up kit (Takara Bio). The products were eluted in 60 μL of nuclease-free water and used for further concentration. An additional purification step was used to eliminate any toxic leftovers from the gel. For this step Agencourt AMPure's SPRI paramagnetic bead technology (Beckman Coulter) was used. The final products were eluted in 10-15 μL of nuclease-free water (to get a final concentration of ˜1-1.5 μg/μL) and used in electroporation experiments.

### Generation of Knock-In T Cells

Primary Human T Cell Culture

Human peripheral blood mononuclear cells (PBMCs) were obtained from whole blood of healthy donors under IRB-approved protocols at St. Jude Children's Research Hospital (SJCRH). To generate gene edited T cells, PBMCs were isolated by Lymphoprep (Abbott Laboratories) gradient centrifugation. CD4+/CD8+ T cells were then enriched from the PBMCs using human anti-CD4- and anti-CD8-specific MicroBeads kit (Miltenyi Biotec) according to the manufacturer's protocol. Enriched T cells were plated in a 24-well non-tissue culture treated plate at 0.5×10⁶ cells/mL in 2 mL T cell media [RPMI (GE Healthcare Life Sciences HyClone Laboratories) containing 10% FBS (GE Healthcare Life Sciences HyClone), and 1% GlutaMAX-I (Invitrogen)]. The next day, selected T cells were stimulated with 25 μL Human T-Activator CD3- and CD28-specific Dynabeads (ThermoFisher) and grown in the T cell media supplemented with recombinant human IL-7 and IL-15 (IL-7: 10 ng/mL; IL-15: 5 ng/mL; PeproTech).

Primary Human T Cell Electroporation

Two days after T cell activation, cells were electroporated to enable site-specific knock-in using Cas9 RNPs. All electroporation experiments were performed on the 4D-Nucleofector™ System X Unit (Lonza) using the EH-115 program. RNPs were pre-complexed at a sgRNA:Cas9 ratio of 4.5:1, prepared by adding 3 μL of 60 μM sgRNA (Synthego) to 1 μL of 40 μL Cas 9 (QB3 Macrolab, University of California, Berkeley) and incubated for 10 min at RT. Complexed RNPs were used right away or frozen for later use. Sequences for all sgRNAs can be found in Table 2. T cells (0.6×10⁶ or 1.0×10⁶) were re-suspended in 17 μL P3 buffer including supplement 1 (Lonza). Subsequently, 4 μL of RNP complex was added together with the dsDNA template donor (2 μg/3 μL unless stated otherwise) and incubated for 10 minutes at room temp. The RNP and dsDNA mix were added to the cell mixture and 23 μL was added to the transfection vessel and electroporated. After electroporation, 80 μL of recovery media (RPMI (GE Healthcare Life Sciences HyClone Laboratories) including 20% FBS (GE Healthcare Life Sciences HyClone), 1% GlutaMAX-I (Invitrogen), IL-7 at 10 ng/mL, and IL-15 at 5 ng/mL) was added to the electroporation vessel. The cells were rested for 30 minutes at 37° C. and 5% CO₂ before being transferred into a 48 well tissue culture plate with 650 μL of recovery media. Two to 3 days after electroporation, the FBS concentration was reduced to 10% in the T cell culture media.

TABLE 2
sgRNA sequences

| sgRNA | Sequence (5′-3′) |
|---|---|
| TRAC | CCCACAGATATCCAGAACCCTG (SEQ ID NO: 9) |
| IL-13 | g12-GAGGAGCGGATGCATAGGCTNGG |
| | (SEQ ID NO: 10) |
| | g10-GGATTGAGGAGCGGATGCATNGG |
| | (SEQ ID NO: 11) |

### Flow Cytometry

Cells were examined by flow cytometry 4 to 6 or >9 days after electroporation to determine the knock-out and knock-in efficiencies of the desired gene constructs. All flow cytometry experiments were performed on the FACSCanto™ instruments (BD Bioscience). FACSDiva (BD Biosciences) and FlowJo v.10 (FlowJo, LLC) were used for analyzing the acquired immunofluorescence data. For surface staining, samples were washed with and stained in PBS (Lonza) with 1% FBS (HyClone). For all experiments, matched isotypes or known negatives (e.g. non-edited or knock-out only T cells) served as gating controls.

Live cells populations were evaluated based on SSC-A over FSC-A gating [35] or using LIVE/DEAD Fixable Aqua Dead Cell Stain Kit (Invitrogen) as a viability dye. TRAC expression was determined by using a mouse anti-human TCRαβ-APC or TCRαβ-PE antibody (BD Biosciences). mClover3, which is protein with a higher fluorescence signal of a jellyfish GFP, positive cells (referred as GFP+ cell in the text) were detected in the GFP channel [19]. Detection of IL13Rα2-CAR was achieved with recombinant human IL13Rα2 protein conjugated to PE (Creative BioMart). Expression of Q8 was detected using anti-human CD34 (Qbend 10) APC antibody (R&D).

### Targeted Deep Sequencing

hTRAC specific amplicons were generated using gene specific primers with partial Illumina adapter overhangs (hTRAC.F—5′-AGTGTAATACCTTGCAGCACCAGAGC-3′ (SEQ ID NO: 12) and hTRAC.R—5′-TTGCTCCAGGCCACAGCACTGTTGC-3′ (SEQ ID NO: 13), overhangs not shown) as previously described [36]. Briefly, hTRAC specific amplicons were generated, indexed, and pooled with other targeted amplicons for other loci. Additionally, 10% PhiX Sequencing Control V3 (Illumina) was added to the pooled amplicon library prior to running the sample on an Illumina Miseq sequencer to generate paired 2×250 reads. Samples were demultiplexed using the index sequences, fastq files were generated, and NGS analysis was performed using CRIS.py [37].

### Analysis of IL-15 Production

5.0×10⁵ T cells were washed with PBS, resuspended in 275 μL of T cell media (RPMI, 10% FBS and 1% GlutaMAX) without cytokines and plated in 96-well V-shaped plates. Cells were then activated with Immunocult™ Human CD3/CD28/CD2 T Cell Activator (Stemcell Technologies) following the manufacturer's protocol and incubated at 37° C. After 24 hours, 250 μL of media was collected from the wells and stored at −80° C. IL-15 were measured using a Human IL-15 Quantikine ELISA Kit (R&D Systems) according to the manufacturer's protocol.

### Statistical Analysis

All statistical analyses were performed in GraphPad PRISM 8 (GraphPad software, Inc.). All experiments were performed at least in duplicates. For comparison between two groups, two-tailed t-test was used. For comparisons of three or more groups, values were log transformed as needed and analyzed by ANOVA. P values <0.05 were considered statistically significant.

### Example 1. Gene Knock-In Using Primary T Cells

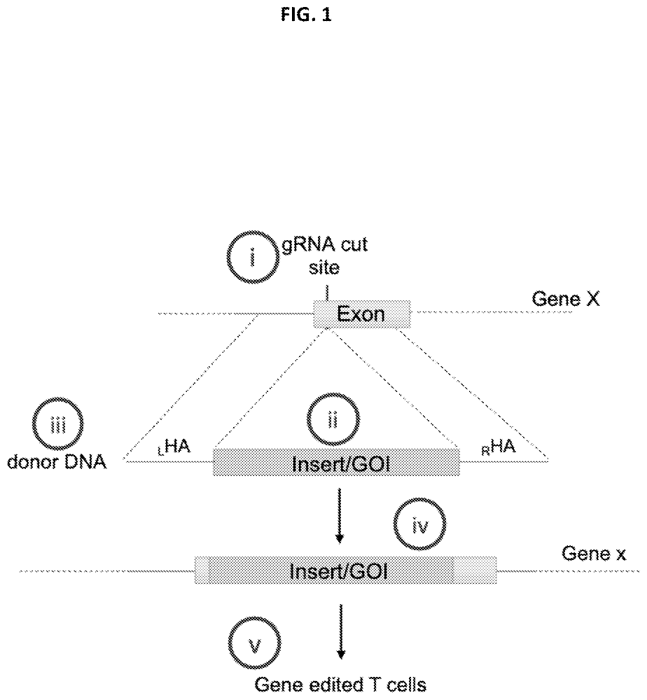

For protocol establishment primary human T cells were chosen as target cells because they are clinically relevant. To optimize knock-in conditions the TRAC locus was targeted for gene insertion, which has been previously explored for the knock-in of several genes [16, 17]. Integration of a promoterless transgene into the TRAC locus will disrupt TRAC expression. However, the endogenous promoter will continue to drive the expression of the newly inserted synthetic gene. For successful integration of a large transgene, the following elements have to be considered: (i) target site and guide RNAs (gRNAs), (ii) transgene design, (iii) donor DNA length, type (ssDNA, dsDNA or plasmid) and delivery, (iv) detection and efficiency of the knock-in and (v) T cell viability (FIG. 1). In the proof-of-concept study, two transgenes were used, IL-15 and mClover3, separated by a 2A sequence. When integrated into the T cell genome, gene edited T cells will express mClover3 fluorescent protein [19] and can be readily detected by flow cytometry (GFP channel). Secretion of IL-15 can be analyzed by ELISA. Importantly, the IL-15 and mClover3 expression cassette is close to the size of a CAR molecule. Hence, the findings can be readily applied for CAR knock-in into human T cells. To optimize the knock-in conditions, template DNA concentration, cell number, homology arm length and knock-in efficiency over time were evaluated, all of which are discussed in detail below. The optimized protocol can result in up to 60% knock-in efficiency and was used to establish guidelines for the gene knock-in in T cells accelerating the process of T cell engineering.

### Example 2. Designing Donor DNA

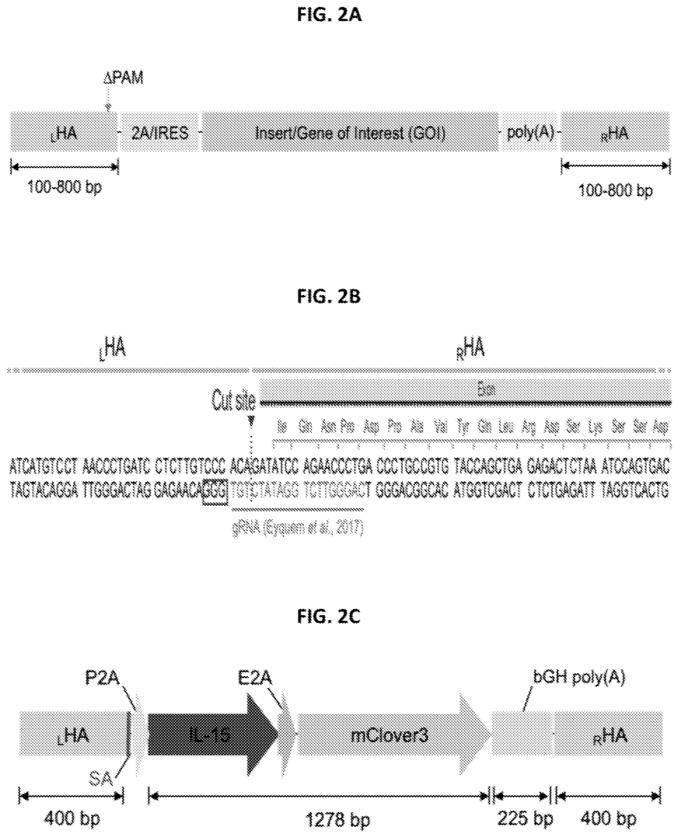

There are no universal guidelines on how to design a donor/template DNA for HDR mediated gene insertion when using CRISPR-Cas9 mediated knock-in. Donor DNA consists of a gene of interest (GOI) flanked by left and right homology arms (_{L}HA and _{R}HA) which are sequences homologous to the target locus (FIG. 2A). In addition, the donor DNA can also include other elements such as a promoter, enhancer, 2A or IRES sequence at the 5′ and poly(A) signal at the 3′ end. HAs are designed to flank the Cas9 cutting site, with equal length HAs of up to 800 bp per side (FIGS. 2A, 2B). Based on these rules, the final donor DNA for the TRAC locus contains the following parts: 400 bp _{L}HA, spliced acceptor (SA) [16], P2A, IL-15, E2A, mClover3, poly(A) (bovine growth hormone polyadenylation signal) and 400 bp _{R}HA (FIG. 2C). A P2A sequence was included at the 5′ end to separate the transgene from a possible fusion to the endogenous gene, and a poly(A) sequence at the 3′ end for efficient termination as simple STOP codon might not be sufficient. Lastly, the PAM sequence was mutated in the _{L}HA to inhibit the Cas9 enzyme from repeatedly cutting the DNA in this location. The construct was then synthesized by GeneArt and inserted into the pMA plasmid. This plasmid was then used as a template for PCR reaction to amplify dsDNA and to generate donor DNA for HDR mediated gene knock-in using CRISPR-Cas9.

### Example 3. Donor DNA Amplification, Purification, and Concentration

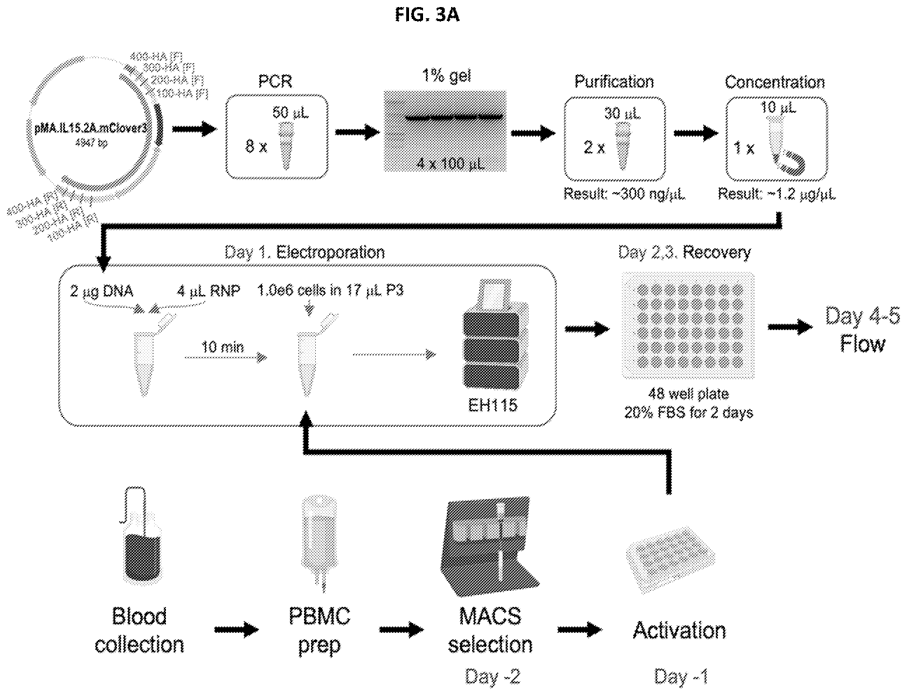

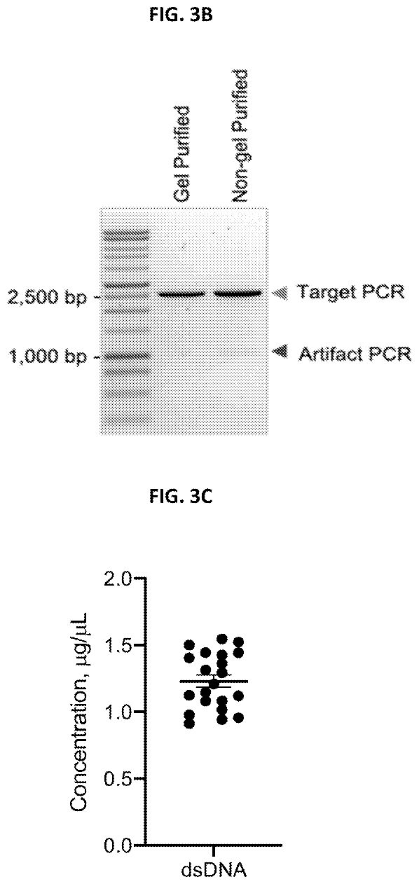

An overview of the donor DNA amplification, purification and concentration protocol is shown in FIG. 3A (upper panel) and detailed protocol is provided in Example 7. Briefly, primers to amplify donor DNA were designed using SnapGene™ for different homology arm lengths (100, 200, 300 and 400 bp) for insertion in the TRAC gene locus. The dsDNA was generated by PCR amplification using CloneAmp HiFi Taq polymerase (Takara Bio), forward and reverse primers, plasmid DNA, and nuclease-free water in a total of 50 μL reaction volume and ran on the ProFlex™ thermocycler (Thermofisher). Two PCR reaction products were combined and separated by electrophoresis on 1% agarose gel for DNA size confirmation and gel purification. To generate high amounts of dsDNA, 8 PCR reactions were run in total and 2 of these reactions were combined in one gel slot.

The amplicons with the size that corresponded to the insert with homology arms size were excised from the gel (FIG. 3B) and gel purified. The products were eluted in total of 60 μL of nuclease-free water. An additional purification step was used to eliminate any toxic leftovers from the gel as well as concentrate dsDNA. For this step, Agencourt AMPure's SPRI paramagnetic beads (Beckman Coulter) were used. The final products were eluted in 10-15 μL of nuclease-free water and used in electroporation experiments. This procedure would routinely yield dsDNA at a concentration of 0.9-1.5 μg/μL (FIG. 3C). At this point, concentrated donor dsDNA was used for the nucleofection (FIG. 3A, middle panel) into activated T cells (FIG. 3A, bottom panel) as described in Materials and Methods section and the protocol (see Example 7).

### Example 4. Optimizing Transgene Knock-In in Primary Human T Cells

After donor DNA was generated, the knock-in efficiency of the IL-15.E2A.mClover3 transgene in primary human T cells was optimized. The following parameters were tested: template dsDNA concentration, cell numbers, homology arm length and recovery time.

### DNA Amount

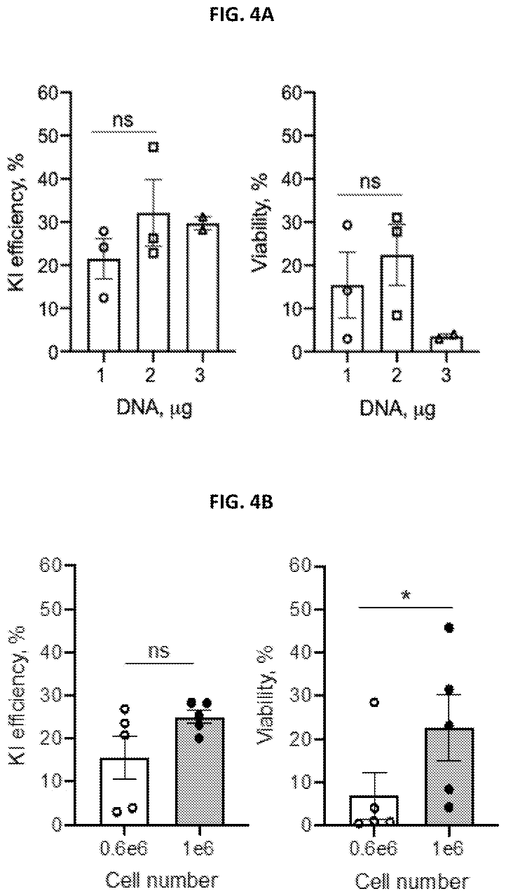

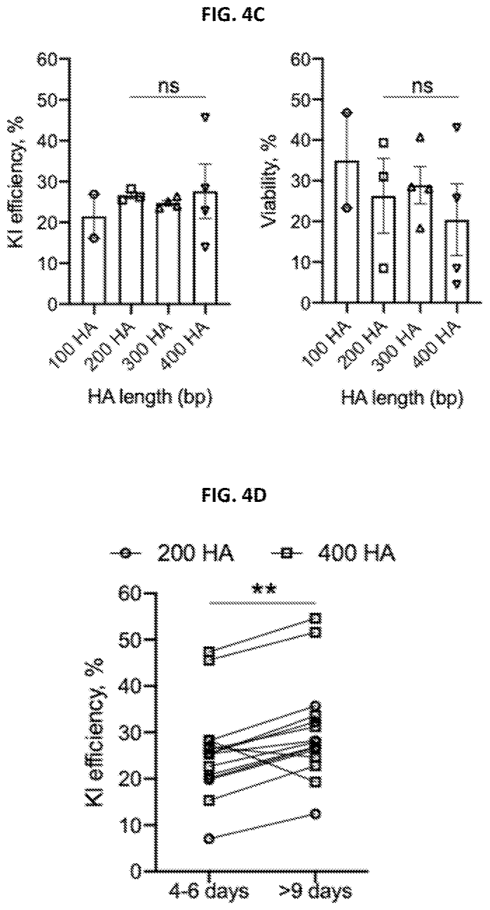

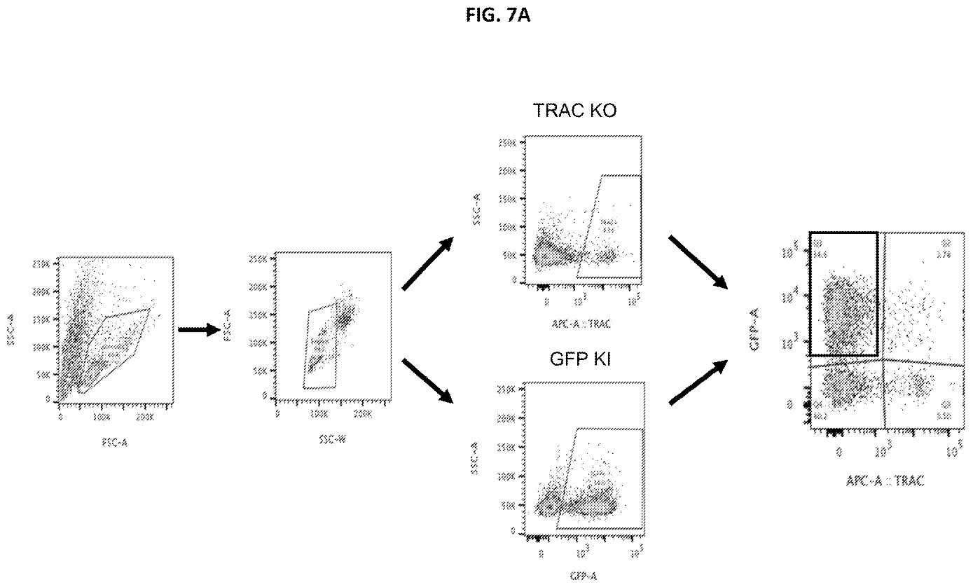

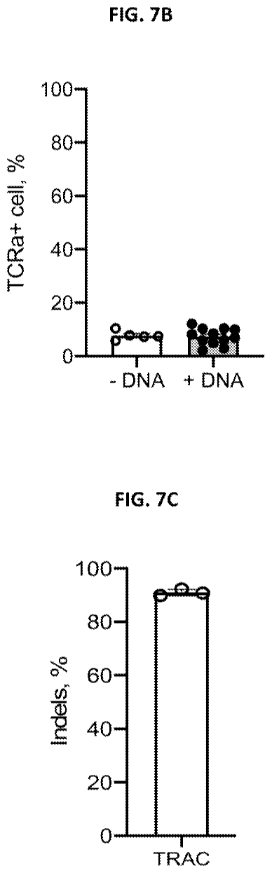

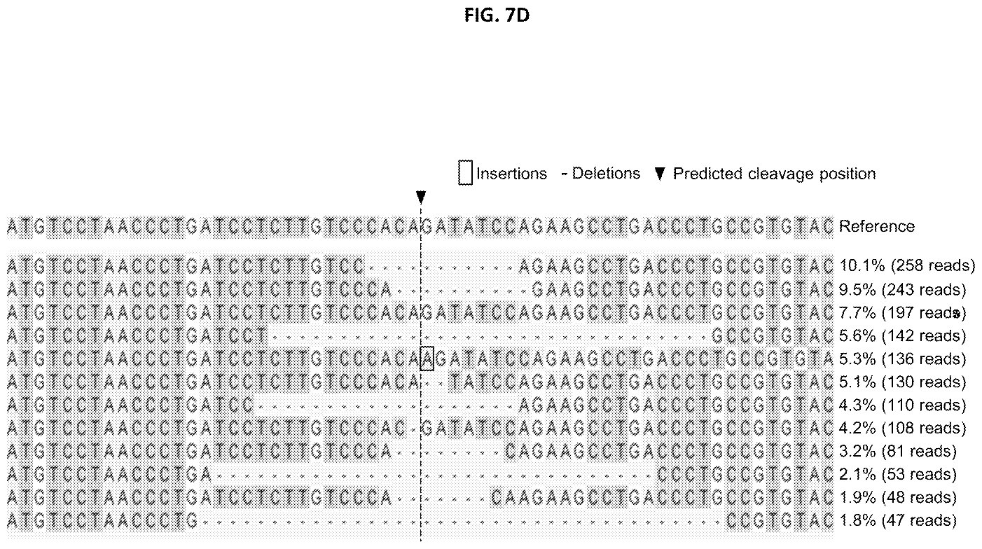

Electroporation of large amounts of plasmid or linear DNA into cells can be toxic resulting in poor cell viability and high rates of cell death [17, 20, 21]. To minimize toxicity while maintaining optimal knock-in efficiencies, the effect of different template DNA quantities was first evaluated. 1 μg, 2 μg or 3 μg of IL-15.E2A.mClover3 DNA was used in 3 μL of nuclease-free water for electroporation together with Cas9:single-guide (sg) RNA ribonucleoproteins (Cas9 RNPs). Electroporation was performed as shown in FIG. 3A (middle panel) and described in the Material and Method section. Briefly, activated T cells were resuspended in P3 electroporation buffer and electroporated with 4 μL of Cas9 RNPs together with 3 μL of donor DNA for HDR. Transgene integration was evaluated 4-6 days later by flow cytometry analysis to determine the percentage of TCRαβ negative and GFP positive T cells (referred to as GFP+ cells thereafter; FIG. 7A). Electroporation of T cells with TRAC specific guide RNA with Cas9 RNPs routinely produced around 91% efficient knock-out of the TRAC gene as shown in FIGS. 7B-7D. As shown in FIG. 4A, 1 μg, 2 μg or 3 μg of template DNA resulted in an average of 21.5%, 32.1%, or 29.7% knock-in efficiency as judged by GFP+ cells (FIG. 4A, left panel) with a cell viability of 15.5%, 22.4%, or 3.6% respectively (FIG. 4A, right panel). Based on this result 2 μg of donor DNA was used in subsequent experiments.

### T Cell Number

Next, it was tested whether increasing the number of T cells per reaction improves T cell recovery post-electroporation and enhance gene editing efficiency. As shown in FIG. 4B, increasing the number of T cells from 0.6×10⁶ to 1.0×10⁶ per electroporation reaction resulted in a higher KI efficiency (15.6% to 25.0%; FIG. 4B). Importantly, increasing T cell numbers per reaction significantly (p=0.04) enhanced T cell survival from 7.0% to 22.6%, respectively (FIG. 4B). As a side note, it was also found that combining two electroporation reaction vessels into one recovery well (48 well plate) further improved T cell viability (data not shown).

### Homology Arms and Recovery Time

It was next evaluated if longer HA length results in improved knock-in efficiency. To test this, the IL-15.E2A.mClover3 transgene was used flanked by 100, 200, 300, 400 bp HAs. As shown in FIG. 4C, an average of 21.5%, 26.6%, 24.8% or 27.7% GFP+ cells was obtained respectively, with no statistically significant difference between groups. In addition, different HA length did not affect T cell viability (FIG. 4C, right panel). Taken together, these results indicate that knock-in efficiency of a large transgene is not dependent on HA length tested.

Finally, knock-in efficiency was evaluated at a later time point (>9 days) post electroporation, which allows T cells to rest, recover and expand. It was found that testing for knock-in efficiency at later time points increased the percentage of GFP+ cells in comparison to early (4-6 days) time points (FIG. 4D).

### Example 5. IL-15 Transgene is Functional

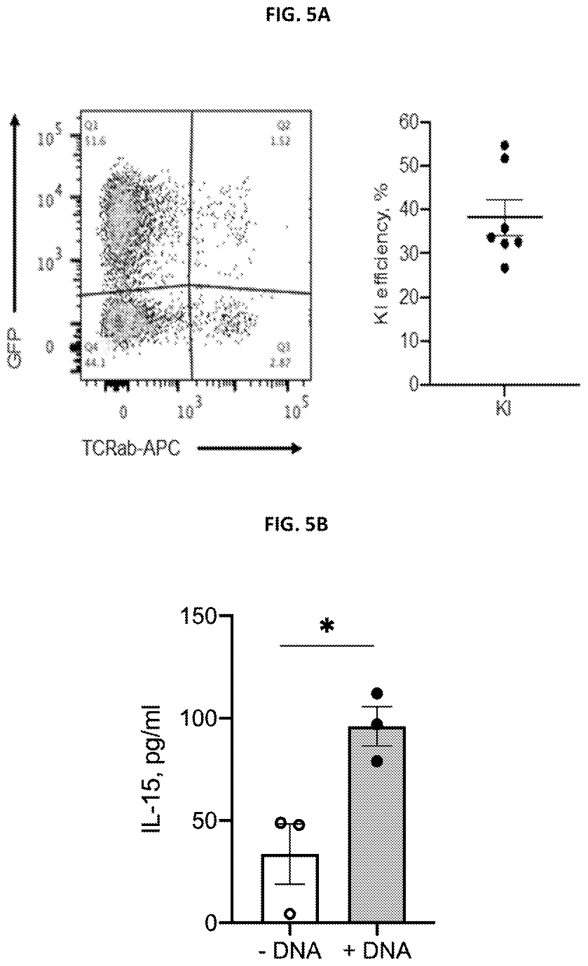

To ensure the robustness of the optimized protocol, three different experimenters performed the knock-in assays, routinely achieving an average of 38.2% KI efficiency (range 26.7-54.6%; FIG. 5A). However, up to this point, gene editing efficiency was evaluated based on GFP+ cells. Since IL-15 is also a component of the transgene, IL-15 secretion was next tested from gene edited T cells via ELISA. Briefly, 8-10 days post electroporation, 5×10⁵ cells were washed with PBS, resuspended in 275 μL of media and plated in 96-well V-shaped plates. After 24 hours, 250 μL of media was collected for IL-15 ELISA. As shown in FIG. 5B, gene edited T cells secreted approximately 2 times as much IL-15 compared to control cells (electroporated without DNA, −DNA).

### Example 6. CAR or BiTE Knock-In into TRAC Locus

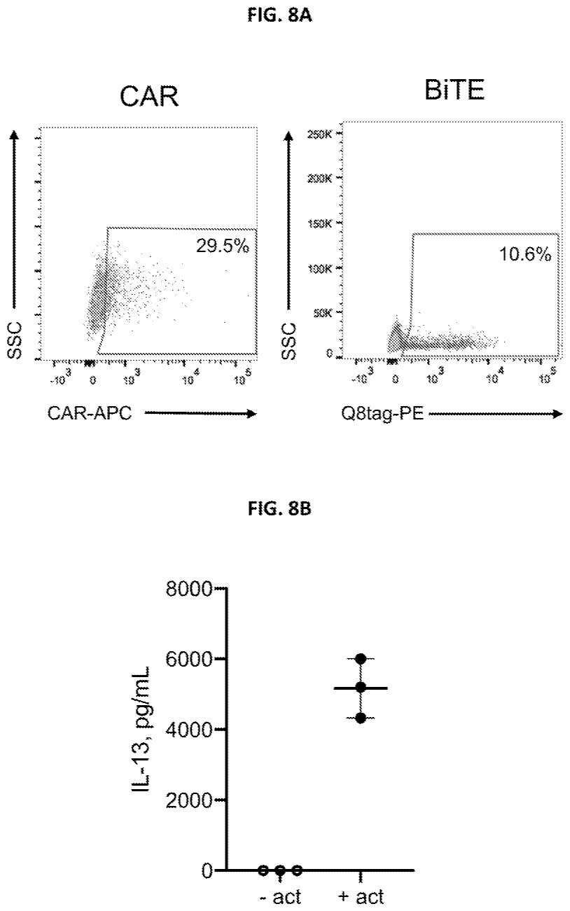

Next it was tested whether a gene encoding a CAR or bispecific T cell engager (BiTE) can be knocked in into the TRAC locus using the established protocols. As shown in FIG. 8A, genes encoding a CAR or BiTE could be knocked in into the TRAC locus with ˜20% efficiency.

### Example 7. IL-15.E2A.mClover3 Knock-In into IL-13 Locus

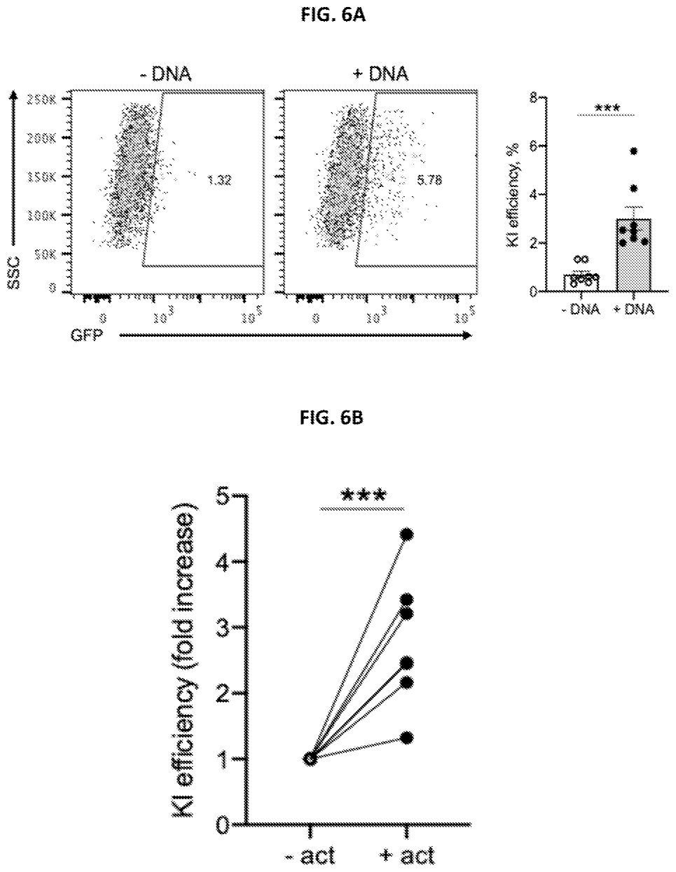

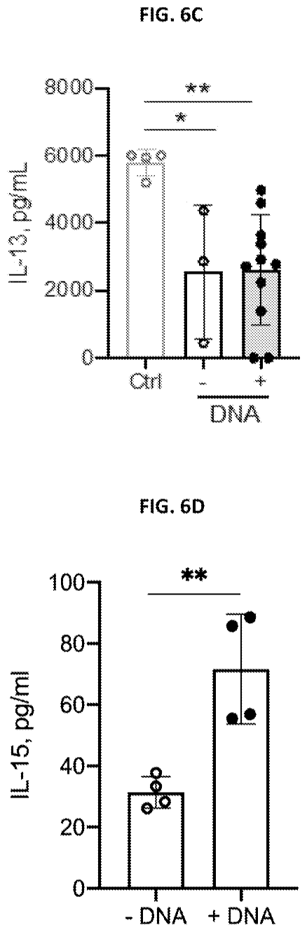

To test this protocol further, the IL-15.E2A.mClover3 transgene knocked in into a different gene locus. For that purpose, the IL-13 locus was picked. It was reasoned that by knocking-in IL-15 into the IL-13 locus, an inducible system can be created as IL-13 is highly secreted upon T-cell activation (FIG. 8B). The same IL-15.E2A.mClover3 transgene was used which was flanked by homology arms for the IL-13 gene locus and CRISPR-Cas9 mediated knock-in experiment was performed using the established guidelines. As shown in FIG. 6A, an average of 3% knock-was achieved as judged by flow cytometry of GFP+ cells. Since IL-13 is activation dependent, it was next tested if expression of the transgene is affected by T cell activation. For that, gene edited T cells were activated with ImmunoCult™ (Human CD3/CD28/CD2 T Cell Activator, Stem Cell Technologies) and GFP+ cells were quantified by flow cytometry 24 hours later. Indeed, an average of about 3-fold improvement was observed in knock-in efficiency as judged by GFP+ cells in activated samples when compared to non-activated T cells (FIG. 6B). In addition, IL-13 gene knock-out +/−donor DNA led to a significant 2.2-fold decrease in IL-13 secretion (p=0.036 ctrl vs −DNA and p=0.007 ctrl vs +DNA), indicating successful IL-13 gene disruption (FIG. 6C). Finally, IL-15 secretion was tested in IL-13 edited and activated T cells. As shown in FIG. 6D, an increase (average of 2.3-fold) in IL-15 secretion was observed from gene edited T cells when compared to control T cells. Thus, not only genes of interest can be knocked in into the regions of choice, but also an inducible system can be created using the optimized protocol.

The following homology arms were used for the IL-13 gene locus:

Left Homology Arm:
(SEQ ID NO: 14)
ACTAAGACTATCTGCTCAGCACTTCTGGTGACCCAAAAGGGTCTGAGGA
CAGGAGCTCAGAGTTGGGTCAGCTGTCCAGGTACTCAGGGTTGTCACAG
GCAAAACTGCTGGAACTCAGGGCAGCATTGCAAATGCCACGCCGCTCTC
AGGGCCCCTTGCCTGCCGCTGGAATTAAACCCACCCAGATCTTGGAAAC
TCTGCCCTGGACCCTTCTCAATAAGTCCATGAGAAATCAAACTCTTTCC
TTTATGCGACACTGGATTTTCCACAAAGTAAAATCAAGATGAGTAAAGA
TGTGGTTTCTAGATAGTGCCTGAAAAAGCAGAGACCATGGTGTCAGGCG
TCACCACTTGGGCCTATAAAAGCTGCCACAAGACGCCAAGGCCACAAGC
CACACAGC
Right Homology Arm:
(SEQ ID NO: 15)
CTATGCATCCGCTCCTCAATCCTCTCCTGTTGGCACTGGGCCTCATGGC
GCTTTTGTTGACCACGGTCATTGCTCTCACTTGCCTTGGCGGCTTTGCC
TCCCCAGGCCCTGTGCCTCCCTCTACAGCCCTCAGGGAGCTCATTGAGG
AGCTGGTCAACATCACCCAGAACCAGAAGGTGAGTGTCGGCTAGCCAGG
GTCCTAGCTATGAGGGCTCCAGGGTGGGTGATTCCCAAGATGAGGTCAT
GAGCAGGCTGGGCCTGGTCCTAAGATGCCTGTAGGTCAGGAAAAATCTC
CATGGACCAAGGCCCGGCCCAGCCATGAGGGAGAGAGGAGCTGGGCTGG
GGGGCTCAGCACTGTGGATGGACCTATGGAGGTGTCTGGCAGACTCCCC
AGGGACTA

### Example 8. CRISPR-Cas9 Knock-In Electroporation Protocol

CRISPR-Cas9 knock-in approaches allow for efficient and fast site-specific gene integration in primary human T cells when donor DNA is provided in non-viral form. This allows for efficient generation of gene edited T cells expressing multiple therapeutically relevant genes. The present disclosure provide guidelines to streamline donor DNA design and maximize editing efficiency for CRISPR-Cas9 gene editing (Table 3).

TABLE 3
Electroporation checklist for large gene knock-in in human T cells

| | |
|---|---|
| sgRNA | 3 μL [60 μM] |
| Cas9 | 1 μL [40 μM] |
| sgRNA:Cas9 (molar ratio) | 4.5:1 |
| RNP incubation | 10 min, RT |
| RNP volume | 4 μL |
| Cells | 1 × 10⁶/17 μL |
| Electroporation solution | P3 + S1 supplement (Lonza) |
| Template | 2 μg dsDNA in 3 μL |
| Homology arm length | 200-400 bp |
| Vol for electroporation | 23 μL |
| Format/program Neon | Strip/EH-115 |
| Incubation after | 30 min at 37 C. (in 80 μL of |
| electroporation | RPMI + 20% FBS + IL7/15) |
| Transfer | 48-well plate (650 μL of |
| | RPMI + 20% FBS + IL7/15) |
| Reactions per well after | 2 |
| electroporation | |

### Knock-In Protocol

The following materials were used in the knock-in protocol:

Biologicals
Healthy donor human blood

Reagents
Lymphoprep (StemCell Technologies; cat. #07801)
HyClone Fetal Bovine Serum (FBS) (Fisher Scientific, SH30071.03, Lot AD20618263)
RPMI-1640 (without L-glutamine; GE Healthcare Life Sciences; cat. #SH30096.01)
GlutaMAX™ (Gibco; cat. #35050061)
PBS (without calcium, magnesium; GE Healthcare Life Sciences; cat. #SH30256.01)
Ethanol, Molecular biology grade (Fisher, cat. #BP2818-100)
Trypan Blue solution (Sigma; cat. #T8154)
IL-7 (Miltenyi Biotec; cat. #130-095-363 or PeproTech; cat. #200-07)
IL-15 (Miltenyi Biotec; cat. #130-095-765 or PeproTech; cat #200-15)
CD4 MicroBeads, human (Miltenyi Biotec; cat. #130-045-101)
CD8 MicroBeads, human (Miltenyi Biotec; cat. #130-045-201)
MACS BSA stock solution (Miltenyi Biotec; cat. #130-091-376)
0.5M EDTA pH8.0 (Life Technology; cat. #15575-038)
P3 Primary Cell 4D-Nucleofector X Kit S (Lonza; cat. #V4XP-3032)
sgRNA targeting TRAC exon1 (Synthego)
Nuclease-free Water (provided with guides, Synthego)
1×TE Buffer pH8.0 (provided with guides, Synthego)
Cas9 enzyme (MacroLab, Berkeley)
Dynabeads Human T-Activator CD3/CD28 (Thermofisher; cat. #11131D)
T Cell TransAct (Miltenyi Biotec; cat. #130-111-160)
HyClone HyPure Water, Molecular Biology Grade (GE Healthcare Life Sciences; cat. #SH30538.01)
ImmunoCult™ Human CD3/CD28/CD2 T Cell Activator (StemCell Technologies; cat. #10970)
NucleoSpin Gel and PCR Clean-up Kit (Takara; cat. #740609.50)
Agencourt AMPure XP (Beckman Coulter; cat. #A63880)
CloneAmp HiFi PCR Premix (Takara; cat. #639298)
Benchtop 1 kb DNA ladder (Promega; cat. #G7541)
Gel Loading Dye, Purple 6× (New England BioLabs; cat. #B7024S)
TopVision Agarose Tablets (Thermo Scientific, cat. #R2801)
UltrPure TAE Buffer 10× (Invitrogen, cat. #15558042)
Mouse anti-human TCRαβ-APC (Clone T10B9.1A-31; BD Biosciences; cat. #563826)
Mouse Isotype Control IgM-APC, κ (Clone G155-228; BD Biosciences; cat. #550883)
P3 Primary Cell 4D-Nucleofector™ X Kit S (Lonza; cat. #V4XP-3032)
Plasmids: custom made from GeneArt

Plasticware and Equipment
24 well tissue culture treated plates (Corning; cat #353047)
48 well tissue culture treated plates (Corning; cat. #353078)
PCR tubes (Applied Biosystems™; cat #A30588)
1.5 mL Eppendorf tubes USA Scientific, cat. #1615-5510)
15 mL Falcon tubes (Fisher Scientific; cat. #14-959-53A)
Flowmi cell strainer (Belart, cat. #H13680-0040)
Falcon Round-Bottom Polystyrene Tubes (Corning, cat. #352057)
MidiMACS Separator (Miltenyi Biotec; cat. #130-042-302)
MACS LS columns (Miltenyi Biotec; cat. #130-042-401)
MACS MultiStand (Miltenyi Biotec; cat. #130-042-303)
MiniMACS Separator (Miltenyi Biotec; cat. #130-042-102)
DynaMag™-Spin Magnet (Thermo Fisher; cat. #12320D)
BD FACSCanto II (BD Biosciences)
Applied Biosystems ProFlex PCR System (Thermo Fisher; cat. #4484073)
NanoDrop™ (Thermo Fisher; cat. #ND-ONEC-W)
4D-Nucleofector™ X Unit (Lonza; cat. #AAF-1002X)
4D-Nucleofector™ Core Unit (Lonza; cat. #AAF-1002B)
Owl™ EasyCast B2 Mini Gel Electrophoresis System (Thermo Scientific™; cat. #B2-BP)
Countess II FL automated cell counter (Invitrogen, cat. #AMQAF1000)
Gel Doc EZ System (Bio-Rad; cat. #1708270)

Software
SnapGene v4 (GSL Biotech)
FlowJo v9 (BD Biosciences)
BD FACSDiva (BD Biosciences)
PRISM v8.4. (GraphPad)

1. Donor DNA Design

Suggestions for donor DNA design are illustrated in FIG. 2. It is recommended to design the homology arms right next to the cut site of the sgRNA of the targeted gene locus. Based on the protocol optimization it is recommended to use homology arms of 400 bp for longer insert sizes. A 2A or IRES sequence should be implemented in front of the transgene to ensure proper separation of the product from the native gene product. When two genes are cloned together in one construct, it is recommended to add a 2A sequence in between those genes to avoid fusion genes during translation. At the end of the transgene, before the 5′ end of the right homology arm, a poly A sequence is beneficial for appropriate gene termination. Lastly, mutating the PAM sequence in the construct inhibits the Cas9 enzyme from repeatedly cutting the DNA in this location.

2. PBMC Preparation

For the studies PBMCs were isolated by Lymphoprep (Abbott Laboratories) gradient centrifugation. Generally, any PBMC isolation method is appropriate for this protocol as long as it produces healthy and viable PBMCs. While cryopreserved PBMCs were used, using fresh PBMCs would result in a higher viability of T cells.

3. T Cell Enrichment

This part of the protocol is a modified version of the MidiMACS kit protocol.
3.1. Thaw a vial of PBMCs (prepared as described above) in a 37° C. water bath. Wash the thawed PBMCs with T cell media (RPMI-1640 media including 10% FBS and 1% GlutaMAX-I). Resuspend the cell-pellet with MACS buffer (500 mL PBS, 25 mL MACS BSA stock solution, and 2 mL 0.5M EDTA pH8.0).
3.2. Count the cells and spin down at 400 g for 5 minutes at room temperature. Aspirate the MACS buffer and add 250 μL of MACS buffer to the cell pellet.
3.3. Add 20 μL of CD4 and 20 μL of CD8 MicroBeads per 10⁷ cells. Ensure proper mixing and incubate for 15 minutes at 4° C.
3.4. After incubating, wash the cells with 2 mL of MACS Buffer and spin down at 400 g for 5 minutes at room temperatures. During the wash step, place the MidiMACS Separator on the MACS MultiStand and a MACS LS column in the MidiMACS Separator and equilibrate the column with 3 mL MACS buffer.
3.5. After washing, aspirate the supernatant and resuspend the stained cells with 500 μL of MACS Buffer. Run the cell suspension through a 40 μM cell strainer to remove dead cell clumps and apply it onto the column.
3.6. Wash the column with 3 mL of MACS buffer. Repeat this step for a total of 3 washes.
3.7. Remove the column from the MidiMACS Separator and place on a 15 mL collection tube. Add 5 mL of MACS buffer and insert the syringe plunger (comes with the MACS LS column) to flush the column and release the cells into the collection tube. Spin at 400 g for 5 minutes at room temperature.
3.8. Add 5 mL of T cell media and count the cells. Plate 10⁶ selected T cells in 2 mL T cell media in one well of a 24 well tissue culture treated plate.
3.9. Rest the plated T cells overnight at 37° C. and 5% CO₂.

4. T Cell Activation

Here three approaches were tested to activate T cell: plate bound CD3/CD28, Dynabeads and Transact. While no differences were observed between Dynabeads and Transact, plate bound CD3/CD28 resulted in lower knock-in efficiencies as well as T cell viability. Thus, two methods for T cell activation were described below.

Dynabeads
4.1. The next day, transfer Dynabeads Human T-Activator CD3/CD28 (25 μL per well of plated T cells) to a 1.5 mL collection tube.
4.2. Add 1 mL MACS buffer to the tube with Dynabeads and place the tube on the back side of a MiniMACS Separator. With the tube pressed against the magnet, remove the MACS buffer.
4.3. Re-suspend the beads in T cell media in the same volume as the volume of Dynabeads in step 4.1.
4.4. Add 25 μL Dynabeads to each well of plated T cells. Add TL-7 at 10 ng/mL and IL-15 at 5 ng/mL.
4.5. Rest the activated T cells for two days at 37° C. and 5% CO₂ before electroporation.

TransAct Media
4.6. The next day, add 28.5 μL T cell TransAct to each 24 well tissue plate with T cells. Add IL-7 at 10 ng/mL and IL-15 at 5 ng/mL.
4.7. Rest the activated T cells for two days at 37° C. and 5% CO₂ before electroporation.

5. HDR Template Preparation
5.1. Design primers to amplify HDR template. (Note: it is recommended to design primers 400 bp away from the Cas9 cut site in order to create 400 bp long homology arms)
5.2. Prepare the PCR mix as listed in Table 4 and keep op ice (8 tubes/reactions are recommended).

TABLE 4
PCR mix per reaction

| | |
|---|---|
| Volume | Reagent |

| | | |
|---|---|---|
| 25 | μL | CloneAmp HiFi PCR Premix |
| 2.5 | μL | Forward primer (10 μM) |
| 2.5 | μL | Reverse primer (10 μM) |
| 1 | μL | Plasmid DNA (15-20 ng) |
| 19 | μL | Nuclease-free water |
| 50 | μL | Total |

5.3. Run the PCR reaction on the Applied Biosystems ProFlex or similar PCR System according to the program in Table 5.

TABLE 5
PCR program (polymerase dependent protocol)

| Step | Temperature | Duration | | Cycles |
|---|---|---|---|---|
| Denature | 98° C. | 30 | seconds | 1 |
| Denature | 98° C. | 10 | seconds | 20 |
| Annealing | +3° C. of | 15 | seconds | |
| | lowest primer Tm | | | |
| Elongation | 72° C. | 5 | seconds/kb | |
| Final elongation | 72° C. | 3 | minutes | 1 |

| | | |
|---|---|---|
| 4° C. | ∞ | 1 |

5.4. Prepare 1% agarose gel. Stain PCR reactions with loading dye 1× and load 2 PCR reactions in one gel well (100 μL PCR reaction total per well). Load Benchtop or similar DNA ladder and run the gel.
5.5. Image the gel and cut out bands with the appropriate band size.
5.6. Perform gel extraction with NucleoSpin Gel and PCR Cleanup kit according manufacturer's protocol. Elute all gel pieces in 2×30 μL nuclease-free water total (use 2 columns and one collection tube).
5.7. Concentrate DNA further with Agencourt AMPure XP beads according to manufacturer's protocol. Elute the final solution from step 5.7. in 10 μL nuclease-free water (recommended final concentration 1-2 μg/μL).
5.8. Measure the final DNA concentration on NanoDrop.

6. Electroporation
6.1. Prepare RNP complexes using a 4.5:1 sgRNA:Cas9 molar ratio (carried out in a RNA-free environment):
   6.1.1. sgRNA working stock preparation: spin down sgRNA tube and add 10 μL 1×TE to make a 150 μM stock. Dilute in 15 μL RNase-free water to generate a 60 μM working stock solution. Transfer 3 μL TRAC exon 1 sgRNA from the 60 μM stock to a PCR tube.
   6.1.2. Add 1 μL cas9 from 40 μM stock to PCR tube containing the sgRNA and incubate for 10 minutes at room temperature (store at −20° C. until use).
6.2. Set up recovery plates for the T cells after electroporation (Note: 2 electroporation reactions of 1×10⁶ cells each combined in one recovery well is recommended for optimal viability and knock-in): 48 well tissue culture treated plate including 550 μL recovery media (RPMI-1640 media including 20% FBS, 1% GlutaMAX-I, IL-7 at 10 ng/mL and IL-15 at 5 ng/mL). The total final volume per well should be 750 μL. (Note: if knock-out efficiency is low, use 0.6×10⁶ cells per electroporation reaction).
6.3. Prepare P3 Primary Cell Nucleofector Solution (17 μL total volume per electroporation reaction): 13.94 μL P3 Primary Cell Nucleofector Solution and 3.06 μL Supplement 1.
6.4. Collect the T cells in a 1.5 mL collection tube and remove the Dynabeads Human T-Activator CD3/CD28 from T cells by pressing the tube against a MiniMACS Separator. Take out the cell solution without the Dynabeads. (Note: if T cells were activated with T cell TransAct, transfer the activated T cells to a collection tube).
6.5. Count the T cells and take 10⁶ cells per electroporation reaction (do not spin cells prior to counting). Spin down the T cells at 200 g for 10 minutes at room temperature.
6.6. Remove all the media from the cell pellet and re-suspend the cell pellet in P3 Primary Cell Nucleofector Solution (1×10⁶ T cells per 17 μL P3 Primary Cell Nucleofector Solution)
6.7. Add 2 μg of HDR template (obtained in step 4.9) in no more than 3 μL nuclease-free water together with 4 μl of RNP (obtained in step 5.1) in a new collection tube. Incubate for 10 minutes at room temperature. (Note: always prepare the following controls: No HDR template+RNP; no HDR template, no RNP; and HDR template, no RNP).
6.8. Add 17 μl of the T cells in P3 Primary Cell Nucleofector Solution to the tube with HDR template and RNP (and to the control tubes).
6.9. Transfer 23 μl of T cells with HDR template and RNP (and of the controls) to one well of the Nucleocuvette Strip and nucleofect the cells using program: EH-115.
6.10. After nucleofection, add 80 μl of recovery media to the T cells in the Nucleocuvette Strip (as per manufacturer's suggestions, do not add the media directly into the well but slowly pipet at the rim of the well).
6.11. Let T cells to recover at 37° C. and 5% CO₂ for 30 minutes. Then transfer the cells to the recovery plate prepared in step 5.2 (2 electroporation reactions into 1 well of the recovery plate).

7. T Cell Expansion
7.1. Incubate the electroporated T cells at 37° C. and 5% CO₂. Split the cells when the media yellows and the cells are at high density (this can take a few days as the cell will grow slowly right after electroporation).
7.2. Dilute the media to 10% FBS using serum-free RPMI-1640 containing 1% GlutaMAX-I only, 2-3 days after electroporation.
7.3. Add IL-7 at 10 ng/mL and IL-15 at 5 ng/mL every 2-3 days.

8. Determine Knock-In
8.1. Determine knock-in efficiency 8-10 days post-electroporation. Detection method is based on the transgene. Detection should be done at a protein level using flow cytometry, ELISA or WB to confirm that the protein is expressed. In addition, targeted next-generation sequencing (NGS) is highly recommended to determine editing efficiency.

As demonstrated in the Examples above, primary human T cells can be engineered to express IL-15 and GFP when integrated into the TRAC locus using CRISPR-Cas9 gene editing and non-viral donor DNA as template. Further, an inducible system was created by inserting IL-15 under the IL-13 promoter to control IL-15 secretion in a T cell activation dependent manner.

The ability to generate T cells expressing a gene-of-interest from a specific locus and/or under a specific promoter opens up new avenues for T cell-based immunotherapies, especially for CAR T cell-based therapies. Currently, CAR T cell products are generated mainly by viral transduction, which poses manufacturing challenges as well as safety concerns due to random integration and potential insertional mutagenesis. The present disclosure provide guidelines on generating template DNA for CRISPR-Cas9 mediated knock-in and perform electroporation to deliver a transgene to the T cells. The level of knock-in efficiency achieved here is sufficient for producing a clinically relevant CAR T cell product.

As synthetic gene integration into the TRAC locus was successful, the established guidelines were then applied to integrate IL-15 into IL-13 locus to potentially design an inducible system. The initial detection of GFP+ cells was very low which was expected given the fact that IL-13 is only expressed/secreted upon T cell activation. When gene edited T cells were activated, an increased percentage of GFP+ cells and increased secretion of IL-15 as assessed by ELISA was observed. However, GFP+ cell number and IL-15 secretion were still relatively low. This in part can be explained by incomplete knock-out of IL-13. Another reason for low efficiency IL-13 locus editing might be the stability of the locus which in part is mediated by chromatin accessibility/structure. To address this, incorporation of insulators or an enhancer might need to be considered when designing donor template.

One hurdle currently limiting the application of existing inducible systems such Syn-Notch and TetON [28-30] is the potential for immunogenicity. By knocking in a transgene into a locus that is expressed only under certain conditions (e.g. IL-13 is only expressed upon T cell activation), it is possible to use knock-in technology as a non-immunogenic inducible transgene expression system.

Multiple variables were tested in the Examples above that were thought to influence T cell editing efficiency, such as homology arm length, DNA concentration, cell numbers and time of recovery post-electroporation. Cell number and time of T cell recovery were shown to be important factors for successful large gene integration. Interestingly, the data indicate that there appears to be no difference in knock-in efficiency when using different length of homology arms. This might be due to the size of homology arms tested. Some differences may be observed in editing efficiencies if longer homology arms (>400 bp) are used, such as 800 bp or 1200 bp. While this might be beneficial for improving editing efficiencies, having longer homology arms may affect DNA concentration which then may lead to a lower T cell viability.

In summary, a reliable protocol was developed to insert genes into the genome of human T cells using CRISPR-Cas9 gene editing and non-viral donor DNA as template. This protocol can be applied for the creation of an inducible expression system.

### REFERENCES

1. Maldini, C. R., G. I. Ellis, and J. L. Riley, CAR T cells for infection, autoimmunity and allotransplantation. Nat Rev Immunol, 2018. 18(10): p. 605-616.
2. Sadelain, M., I. Riviere, and S. Riddell, Therapeutic T cell engineering. Nature, 2017. 545(7655): p. 423-431.
3. Maude, S. L., et al., Chimeric antigen receptor T cells for sustained remissions in leukemia. N. Engl. J. Med., 2014. 371(16): p. 1507-1517.
4. Davila, M. L., et al., Efficacy and toxicity management of 19-28z CAR T cell therapy in B cell acute lymphoblastic leukemia. Sci. Transl. Med., 2014. 6(224): p. 224ra25.
5. Lee, D. W., et al., T cells expressing CD19 chimeric antigen receptors for acute lymphoblastic leukaemia in children and young adults: a phase 1 dose-escalation trial. Lancet, 2015. 385(9967): p. 517-28.
6. Hacein-Bey-Abina, S., et al., LMO2-associated clonal T cell prolferation in two patients after gene therapy for SCID-XI. Science, 2003. 302(5644): p. 415-419.
7. Nam, C. H. and T. H. Rabbitts, The role of LMO2 in development and in T cell leukemia after chromosomal translocation or retroviral insertion. Mol Ther, 2006. 13(1): p. 15-25.
8. van der Loo, J. C. and J. F. Wright, Progress and challenges in viral vector manufacturing. Hum Mol Genet, 2016. 25(R1): p. R42-52.
9. Merten, O. W., et al., Large-scale manufacture and characterization of a lentiviral vector produced for clinical ex vivo gene therapy application. Hum Gene Ther, 2011. 22(3): p. 343-56.
10. Wright, J. F., Manufacturing and characterizing AAV-based vectors for use in clinical studies. Gene Ther, 2008. 15(11): p. 840-8.
11. David, R. M. and A. T. Doherty, Viral Vectors: The Road to Reducing Genotoxicity. Toxicol Sci, 2017. 155(2): p. 315-325.
12. Tyagaraj an, S., et al., Autologous cryopreserved leukapheresis cellular material for chimeric antigen receptor-T cell manufacture. Cytotherapy, 2019. 21(12): p. 1198-1205.
13. Schumann, K., et al., Generation of knock-in primary human T cells using Cas9 ribonucleoproteins. Proc Natl Acad Sci USA, 2015. 112(33): p. 10437-42.
14. Xu, X., et al., Efficient homology-directed gene editing by CRISPR Cas9 in human stem and primary cells using tube electroporation. Sci Rep, 2018. 8(1): p. 11649.
15. Hale, M., et al., Homology-Directed Recombination for Enhanced Engineering of Chimeric Antigen Receptor T Cells. Mol Ther Methods Clin Dev, 2017. 4: p. 192-203.
16. Eyquem, J., et al., Targeting a CAR to the TRAC locus with CRISPR Cas9 enhances tumour rejection. Nature, 2017. 543ζ 7643): p. 113-117.
17. Roth, T. L., et al., Reprogramming human T cell function and specificity with non-viral genome targeting. Nature, 2018. 559(7714): p. 405-409.
18. MacLeod, D. T., et al., Integration of a CD19 CAR into the TCR Alpha Chain Locus Streamlines Production of Allogeneic Gene-Edited CAR T Cells. Mol Ther, 2017. 25(4): p. 949-961.
19. Bajar, B. T., et al., Improving brightness and photostability of green and red fluorescent proteins for live cell imaging and FRET reporting. Sci Rep, 2016. 6: p. 20889.
20. Cornu, T. I., C. Mussolino, and T. Cathomen, Refining strategies to translate genome editing to the clinic. Nat Med, 2017. 23(4): p. 415-423.
21. Luecke, S., et al., cGAS is activated by DNA in a length-dependent manner. EMBO Rep, 2017. 18(10): p. 1707-1715.
22. Nguyen, D. N., et al., Polymer-stabilized Cas9 nanoparticles and modified repair templates increase genome editing efficiency. Nat Biotechnol, 2020. 38(1): p. 44-49.
23. Kan, Y., et al., Mechanisms of precise genome editing using oligonucleotide donors. Genome Res, 2017. 27(7): p. 1099-1111.
24. Liu, M., et al., Methodologies for Improving HDR Efficiency. Front Genet, 2018. 9: p. 691.
25. Paix, A., et al., Precision genome editing using synthesis-dependent repair of Cas9-induced DNA breaks. Proc Natl Acad Sci USA, 2017. 114(50): p. E10745-E10754.
26. Sadelain, M., E. P. Papapetrou, and F. D. Bushman, Safe harbours for the integration of new DNA in the human genome. Nat Rev Cancer, 2011. 12(1): p. 51-8.
27. Odak, A., et al., 2019 ASGCT Abstract Book. Abstract 941: Novel Genomic Safe Harbors for Effective CAR T Cell Engineering. Molecular Therapy, 2019. 27(4S1): p. 1.
28. Morsut, L., et al., Engineering Customized Cell Sensing and Response Behaviors Using Synthetic Notch Receptors. Cell, 2016. 164(4): p. 780-91.
29. Roybal, K. T., et al., Engineering T Cells with Customized Therapeutic Response Programs Using Synthetic Notch Receptors. Cell, 2016. 167(2): p. 419-432 e16.
30. Das, A. T., L. Tenenbaum, and B. Berkhout, Tet-On Systems For Doxycycline-inducible Gene Expression. Curr Gene Ther, 2016. 16(3): p. 156-67.
31. Krenciute, G., et al., Characterization and Functional Analysis of scFv-based Chimeric Antigen Receptors to Redirect T Cells to IL13Ralpha2-positive Glioma. Mol Ther, 2016. 24(2): p. 354-363.
32. Chaudhary, A., et al., TEM8/ANTXR1 blockade inhibits pathological angiogenesis and potentiates tumoricidal responses against multiple cancer types. Cancer Cell, 2012. 21(2): p. 212-226.
33. Williams, L., et al. 31st Annual Meeting and Associated Programs of the Society for Immunotherapy of Cancer (SITC 2016): P51 T cells redirected to TEM8 have antitumor activity but induce ‘on target off cancer toxicity’ in preclinical models. in Journal for immunotherapy of cancer. 2016. Springer.
34. Iwahori, K., et al., Engager T cells: a new class of antigen-specific T cells that redirect bystander T cells. Mol Ther, 2015. 23(1): p. 171-8.
35. Bohmer, R. M., E. Bandala-Sanchez, and L. C. Harrison, Forward light scatter is a simple measure of T-cell activation and proliferation but is not universally suited for doublet discrimination. Cytometry A, 2011. 79(8): p. 646-52.
36. Sentmanat, M. F., et al., A Survey of Validation Strategies for CRISPR-Cas9 Editing. Sci Rep, 2018. 8(1): p. 888.
37. Connelly, J. P. and S. M. Pruett-Miller, CRIS.py: A Versatile and High-throughput Analysis Program for CRISPR-based Genome Editing. Sci Rep, 2019. 9(1): p. 4194.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of genetically modifying an immune cell, comprising introducing into the immune cell at least one transgene, wherein the at least one transgene is inserted at the interleukin 13 (IL-13) gene locus within the immune cell genome.

2. The method of claim 1, wherein the at least one transgene is inserted such that expression of the at least one transgene is under control of an endogenous IL-13 promoter within the immune cell genome.

3. The method of claim 1, wherein expression of the immune cell's endogenous IL-13 is reduced or abolished.

4. The method of claim 1, wherein the at least one transgene encodes a therapeutic molecule.

5. The method of claim 4, wherein the therapeutic molecule is selected from a chimeric antigen receptor (CAR), a cytokine, a cytokine receptor, a chimeric cytokine receptor, a switch receptor, a chemokine, an antibody, and a bispecific antibody.

6. The method of claim 4, wherein the therapeutic molecule is a CAR, a cytokine, or a bispecific T cell engager (BiTE).

7-11. (canceled)

12. The method of claim 1, wherein the at least one transgene is operatively linked to one or more of the following:
1) at its 5′ end, a sequence encoding a self-cleaving peptide and/or an internal ribosomal entry site (IRES);
2) at its 3′ end, a polyadenylation (polyA) sequence; and/or
3) at least one insulator and/or enhancer sequence.

13. The method of claim 12, wherein the self-cleaving peptide is a 2A peptide.

14-15. (canceled)

16. The method of claim 1, wherein the insertion of the at least one transgene is mediated by a site-specific nuclease.

17. The method of claim 16, wherein the site-specific nuclease comprises a Cas protein and a guide RNA capable of targeting the IL-13 gene locus.

18. The method of claim 17, wherein the Cas protein is a Cas9 protein.

19. (canceled)

20. The method of claim 17, wherein the guide RNA comprises the nucleotide sequence of SEQ ID NO: 16 or SEQ ID NO: 17, or a nucleotide sequence having at least 80% identity thereof.

21. The method of claim 1, wherein the at least one transgene is introduced into the immune cell via a donor polynucleotide.

22. (canceled)

23. The method of claim 21, wherein the donor polynucleotide is a single-stranded DNA, a double-stranded DNA, or a plasmid.

24. (canceled)

25. The method of claim 21, wherein the donor polynucleotide comprises a 5′ homology arm and a 3′ homology arm, and wherein the 5′ homology arm and the 3′ homology arm comprise sequences flanking the IL-13 gene locus.

26-28. (canceled)

29. The method of claim 25any, wherein the 5′ homology arm in the donor polynucleotide comprises the nucleotide sequence of SEQ ID NO: 14, or a nucleotide sequence having at least 80% identity thereof, or a fragment thereof; and/or
the 3′ homology arm in the donor polynucleotide comprises the nucleotide sequence of SEQ ID NO: 15, or a nucleotide sequence having at least 80% identity thereof, or a fragment thereof.

30-36. (canceled)

37. The method of claim 1, said method further comprises activating the immune cell to increase expression of the at least one transgene.

38. (canceled)

39. The method of claim 1, wherein the immune cell is a T cell.

40. The method of claim 39, wherein the T cell is an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (TSCM), a naïve T-cell, an effector T-cell, a T-helper cell, or a regulatory T-cell (Treg).

41. (canceled)

42. The method of claim 1, wherein the immune cell is a natural killer (NK) cell.

43-46. (canceled)

47. A genetically modified immune cell prepared according to the method of claim 1.

48. A genetically modified immune cell, comprising at least one transgene inserted at the interleukin 13 (IL-13) gene locus within the immune cell genome.

49-68. (canceled)

69. A method of genetically modifying an immune cell, comprising introducing into the immune cell at least one transgene, wherein the at least one transgene is inserted at the granulocyte-macrophage colony-stimulating factor (GM-CSF) gene locus within the immune cell genome.

70-111. (canceled)

112. A genetically modified immune cell prepared according to the method of claim 69.

113. A genetically modified immune cell, comprising at least one transgene inserted at the granulocyte-macrophage colony-stimulating factor (GM-CSF) gene locus within the immune cell genome.

114-133. (canceled)

134. A pharmaceutical composition comprising the genetically modified immune cell of claim 47, and a pharmaceutically acceptable carrier.

135. A method of treating a disease in a subject in need thereof, comprising administering to the subject a therapeutically effective amount of the genetically modified immune cell of claim 47, or a pharmaceutical composition thereof.

136-138. (canceled)
